(19) United States
(12) Patent Application Publication
(10) Pub. No.: US 2006/0263293 A1
(43) Pub. Date: Nov. 23, 2006

(54) Click chemistry method for synthesizing molecular imaging probes

(76) Inventors: Hartmuth Kolb, Playa Del Rey, CA (US); Joseph C. Walsh, Pacific Palisades, CA (US); Kai Chen, Los Angeles, CA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/413,596

(22) Filed: Apr. 27, 2006

Related U.S. Application Data
(60) Provisional application No. 60/675,267, filed on Apr. 27, 2005.

(51) Int. Cl. A61K 51/00; C07F 5/00

(52) U.S. Cl. 424/1.49; 424/1.69; 530/400; 534/11

(57) ABSTRACT

The present disclosure provides a method for preparing a radioactive ligand or radioactive substrate having affinity for a target biomacromolecule, the method comprising: (a) reacting a first compound comprising a first functional group capable of participating in a click chemistry reaction, with a radioactive reagent under conditions sufficient to displace the leaving group with a radioactive component of the radioactive reagent to form a first radioactive compound; (b) providing a second compound comprising a second complementary functional group capable of participating in a click chemistry reaction with the first functional group; (c) reacting the first functional group of the first radioactive compound with the complementary functional group of the second compound via a click chemistry reaction to form the radioactive ligand or substrate; and (d) isolating the radioactive ligand or substrate.

### CROSS REFERENCE TO RELATED APPLICATIONS

This application claims the benefit of U.S. Provisional Application No. 60/675,267 , filed Apr. 27, 2005, which is incorporated herein in its entirety.

### FIELD OF THE INVENTION

The invention relates to the use of click chemistry methods for preparing high affinity molecular imaging probes, particularly PET imaging probes.

### BACKGROUND OF THE INVENTION

Positron Emission Tomography (PET) is a molecular imaging technology that is increasingly used for detection of disease. PET imaging systems create images based on the distribution of positron-emitting isotopes in the tissue of a patient. The isotopes are typically administered to a patient by injection of probe molecules that comprise a positron-emitting isotope, such as F-18, C-11, N-13, or O-15, covalently attached to a molecule that is readily metabolized or localized in the body (e.g., glucose) or that chemically binds to receptor sites within the body. In some cases, the isotope is administered to the patient as an ionic solution or by inhalation. One of the most widely used positron-emitter labeled PET molecular imaging probes is 2-deoxy-2-[¹⁸F]fluoro-D-glucose ([¹⁸F]FDG).

PET scanning using the glucose analog [¹⁸F]FDG, which primarily targets glucose transporters, is an accurate clinical tool for the early detection, staging, and restaging of cancer. PET-FDG imaging is increasingly used to monitor cancer chemo- and chemoradiotherapy, because early changes in glucose utilization have been shown to correlate with outcome predictions. A characteristic feature of tumor cells is their accelerated glycolysis rate, which results from the high metabolic demands of rapidly proliferating tumor tissue. Like glucose, FDG is taken up by cancer cells via glucose transporters and is phosphorylated by hexokinase to FDG-6 phosphate. The latter cannot proceed any further in the glycolysis chain, or leave the cell due to its charge, allowing cells with high glycolysis rates to be detected.

Although useful in many contexts, limitations of FDG-PET imaging for monitoring cancer exist as well. Accumulation in inflammatory tissue limits the specificity of FDG-PET. Conversely, nonspecific FDG uptake may also limit the sensitivity of PET for tumor response prediction. Therapy induced cellular stress reactions have been shown to cause a temporary increase in FDG-uptake in tumor cell lines treated by radiotherapy and chemotherapeutic drugs. Further, physiological high normal background activity (i.e., in the brain) can render the quantification of cancer-related FDG-uptake impossible in some areas of the body.

Due to these limitations, other PET imaging tracers are being developed to target other enzyme-mediated transformations in cancer tissue, such as 6-[F-18]fluoro-L-DOPA for dopamine synthesis, 3′-[F-18]Fluoro-3′-deoxythymidine (FLT) for DNA replication, and [C-11](methyl)choline for choline kinase, as well as ultra high-specific activity receptor-ligand binding (e.g., 16α [F-18]fluoroestradiol) and potentially gene expression (e.g., [F-18]fluoro-ganciclovir). Molecularly targeted agents have demonstrated great potential value for non-invasive PET imaging in cancers.

These studies have demonstrated the great value of non-invasive PET imaging for specific metabolic targets of cancer. Ongoing research efforts are directed to identifying additional biomarkers that show a very high affinity to, and specificity for, tumor targets to support cancer drug development and to provide health care providers with a means to accurately diagnose disease and monitor treatment. Such imaging probes can dramatically improve the apparent spatial resolution of the PET scanner, allowing smaller tumors to be detected, and nanomole quantities to be injected in patients.

Traditional ¹⁸F-labeling of small molecules to form PET imaging probes involves displacement of a suitably activated precursor with [18F]fluoride in a compatible reaction media, such as acetonitrile. [18F]fluoride attachment occurs via nucleophilic displacement of substituted sulfonate or nitro moieties, usually at elevated temperatures. Under such reaction conditions, the reactivity of [18F]fluoride may be limited by sterics and electronic effects inherent in the target molecule. To complicate matters further, the use of protecting groups may also be needed to enhance the overall yield of the labeled material usually by preventing unwanted side reactions. The selection of protecting groups must be evaluated on a case-by-case basis and their effect, good or otherwise, must be determined experimentally. In order to prepare a large number of [18F]-labeled compounds, every precursor must contain a leaving group as well as optimized protecting groups. Thus, this strategy is not general enough for quickly modifying candidate imaging probes to optimize their physiochemical, pharmacokinetic, and efficacy properties.

There is a need in the art for an improved method for quickly synthesizing imaging probes that avoids the problems of the prior art, such as the need for optimized protecting groups.

### SUMMARY OF THE INVENTION

The present invention utilizes click chemistry to provide a more efficient method for labeling molecules with a radioactive isotope. The method of the invention is characterized by reactive partners, mild coupling conditions, generality towards coupling over a wide range of compounds, and high reaction specificity, also referred to as chemical orthogonality, such that the need for protecting groups is eliminated and a larger population of molecules may undergo facile radiolabeling.

In one aspect, the inventive method involves reaction of a reactive precursor (e.g., a small molecule or a biomolecule) bearing a functional group known to participate in click chemistry reactions (Kolb, H. C.; Finn, M. G.; Sharpless, K. B. Angewandte Chemie, International Edition 2001, 40, 2004-2021) with a radioactive precursor molecule comprising a radioactive isotope covalently attached to a complementary functional group also known to participate in click chemistry reactions. In a preferred embodiment, the paired functional groups of the precursor molecules are an alkyne and an azide, meaning one precursor carries an alkynyl functional group and the other carries an azide, which quickly react in the presence of a metal salt, such as copper acetate, which catalyzes the coupling under mild reaction conditions.

In one embodiment, the inventive method involves a click chemistry reaction between two precursor molecules and a reactive group capable of participating in a click chemistry reaction. One or both of the precursor molecules may further include a linkage between the group and the click chemistry functional group. One of the precursor molecules also comprises a leaving group that can be readily displaced in a nucleophilic substitution reaction. The leaving group is displaced by a radioisotope, such as F-18, and the two functional groups are reacted to covalently link the two precursor molecules, thus forming a radioactive compound, or molecular imaging probe, that can, for example, allow in vivo diagnosis and identification of a tumor, and provide mechanistic information on tumor type for treatment.

In a preferred ligand embodiment, the invention is a method for preparing a radioactive ligand or radioactive substrate having affinity for a target biomacromolecule, the method comprising:

(a) reacting a first compound comprising i) a first molecular structure; ii) a leaving group; iii) a first functional group capable of participating in a click chemistry reaction; and optionally, iv) a linker between the first functional group and the molecular structure, with a radioactive reagent under conditions sufficient to displace the leaving group with a radioactive component of the radioactive reagent to form a first radioactive compound;

(b) providing a second compound comprising i) a second molecular structure; ii) a second complementary functional group capable of participating in a click chemistry reaction with the first functional group, wherein the second compound optionally comprises a linker between the second compound and the second functional group;

(c) reacting the first functional group of the first radioactive compound with the complementary functional group of the second compound via a click chemistry reaction to form the radioactive ligand or substrate; and

(d) isolating the radioactive ligand or substrate.

In a preferred embodiment, the biological target molecule is an enzyme such as thymidine kinase. The radioactive isotope is preferably fluorine-18 fluoride in the form of a coordination compound comprising a phase transfer catalyst and salt complex. Exemplary leaving groups include halogens, pseudohalogens, the nitro moiety, diazonium salts and sulfonate esters. Non-exclusive examples of leaving groups may include sulfonoxy group (methanesulfonyl, trifluomethanesulfonyl, tolylsulfonyl, 4 -nitrobenzenesulfonyl, 4-bromobenzenesulfonyl), diazonium salts, the nitro group and halo group, including iodo, bromo and chloro.

### DETAILED DESCRIPTION OF THE PREFERRED EMBODIMENTS

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

I. Definitions

As used herein, the singular forms “a”, “an”, “the”, include plural referents unless the context clearly dictates otherwise.

“Alkyl” refers to a hydrocarbon chain, typically ranging from about 1 to 20 atoms in length. Such hydrocarbon chains may be branched or straight chain, although typically straight chain is preferred. Exemplary alkyl groups include ethyl, propyl, butyl, pentyl, 1-methylbutyl, 1-ethylpropyl, 3-methylpentyl, and the like. As used herein, “alkyl” includes cycloalkyl when three or more carbon atoms are referenced.

“Anchor site” as used herein is synonymous with the first binding site.

“Aryl” means one or more aromatic rings, each of 5 or 6 core carbon atoms. Aryl includes multiple aryl rings that may be fused, as in naphthyl or unfused, as in biphenyl. Aryl rings may also be fused or unfused with one or more cyclic hydrocarbon, heteroaryl, or heterocyclic rings. As used herein, “aryl” includes heteroaryl.

A “biological target” can be any biological molecule involved in biological pathways associated with any of various diseases and conditions, including cancer (e.g., leukemia, lymphomas, brain tumors, breast cancer, lung cancer, prostate cancer, gastric cancer, as well as skin cancer, bladder cancer, bone cancer, cervical cancer, colon cancer, esophageal cancer, eye cancer, gallbladder cancer, liver cancer, kidney cancer, laryngeal cancer, oral cancer, ovarian cancer, pancreatic cancer, penile cancer, glandular tumors, rectal cancer, small intestine cancer, sarcoma, testicular cancer, urethral cancer, uterine cancer, and vaginal cancer), diabetes, neurodegenerative diseases, cardiovascular diseases, respiratory diseases, digestive system diseases, infectious diseases, inflammatory diseases, autoimmune diseases, and the like. Exemplary biological pathways include, for example, cell cycle regulation (e.g., cellular proliferation and apoptosis), angiogenesis, signaling pathways, tumor suppressor pathways, inflammation (COX-2), oncogenes, and growth factor receptors. The biological target may also be referred to as the “target biomacromolecule” or the “biomacromolecule.” The biological target can be a receptor, such as enzyme receptors, ligand-gated ion channels, G-protein-coupled receptors, and transcription factors. The biologically target is preferably a protein or protein complex, such as enzymes, membrane transport proteins, hormones, and antibodies. In one particularly preferred embodiment, the protein biological target is an enzyme, such as carbonic anhydrase-II and its related isozymes such as carbonic anhydrase IX and XII.

“Complementary functional groups” as used herein, means chemically reactive groups that react with one another with high specificity (i.e., the groups are selective for one another and their reaction provides well-defined products in a predictable fashion) to form new covalent bonds.

“Cycloalkyl” refers to a saturated or unsaturated cyclic hydrocarbon chain, including bridged, fused, or spiro cyclic compounds, preferably made up of 3 to about 12 carbon atoms, more preferably 3 to about 8.

“Heteroaryl” is an aryl group containing from one to four heteroatoms, preferably N, O, or S, or a combination thereof. Heteroaryl rings may also be fused with one or more cyclic hydrocarbon, heterocyclic, aryl, or heteroaryl rings.

“Heterocycle” or “heterocyclic” means one or more rings of 5-12 atoms, preferably 5-7 atoms, with or without unsaturation or aromatic character and having at least one ring atom which is not a carbon. Preferred heteroatoms include sulfur, oxygen, and nitrogen.

A “kinase” as used herein and also defined as well known in the art, is an enzyme that transfers a phosphate from adenosine triphosphate (ATP) onto a substrate molecule. A kinase includes a binding site for ATP, which is a cofactor in the phosphorylation, and at least one binding site for the substrate molecule, which is typically another protein.

“Leaving group”, as used herein refers to groups that are readily displaced, for example, by a nucleophile, such as an amine, a thiol or an alcohol nucleophile or its salt. Such leaving groups are well known and include, for example carboxylates, N-hydroxysuccinimide, N-hydroxybenzotriazole, halides, triflates, tosylates, —OR and —SR and the like.

A “ligand” is a molecule, preferably having a molecular weight of less than about 800 Da., more preferably less than about 600 Da., comprising a first group exhibiting affinity for a first binding site on a biological target molecule, such as a protein, and a second group exhibiting affinity for a second binding site on the same biological target molecule. The two binding sites can be separate areas within the same binding pocket on the target molecule. The ligands preferably exhibit nanomolar binding affinity for the biological target molecule. In certain aspects as disclosed herein, a ligand is used interchangeably with a “substrate.” A ligand may comprise a “molecular structure” as defined herein.

A “linker” as used herein refers to a chain comprising 1 to 10 atoms and may comprise of the atoms or groups, such as C, —NR—, O, S, —S(O)—, —S(O)₂—, CO, —C(NR)— and the like, and wherein R is H or is selected from the group consisting of (C₁₋₁₀)alkyl, (C₃₋₈)cycloalkyl, aryl(C₁₋₅)alkyl, heteroaryl(C₁₋₅)alkyl, amino, aryl, heteroaryl, hydroxy, (C₁₋₁₀)alkoxy, aryloxy, heteroaryloxy, each substituted or unsubstituted. The linker chain may also comprise part of a saturated, unsaturated or aromatic ring, including polycyclic and heteroaromatic rings.

A “metal chelating group” as used herein, is as defined in the art, and may include, for example, a molecule, fragment or functional group that selectively attaches or binds metal ions, and forms a complex. Certain organic compounds may form coordinate bonds with metals through two or more atoms of the organic compound. Examples of such molecule include DOTA, EDTA, and porphine.

“Molecular structure” refers to a molecule or a portion or fragment of a molecule that is attached to the click functional group, optionally attached to a leaving group and/or radioactive isotope or, in certain variations, the molecule may be attached to a linker that is attached to the click functional group. Non-exclusive examples of such molecular structures include, for example, a substituted or unsubstituted methylene, alkyl groups (C1-C10) that are linear or branched, each optionally comprising a heteroatoms selected from the group consisting of O, N and S, aryl and heteroaryl groups each unsubstituted or substituted, biomacromolecules, nucleosides and their analogs or derivatives, peptides and peptide mimics, carbohydrates and combinations thereof.

“Polydentate metal chelating group” means a chemical group with two or more donator atoms that can coordinate to (i.e. chelate) a metal simultaneously. Accordingly, a polydentate group has two or more donor atoms and occupies two or more sites in a coordination sphere.

The terms “patient” and “subject” refer to any human or animal subject, particularly including all mammals.

The term “pericyclic reaction” refers to a reaction in which bonds are made or broken in a concerted cyclic transition state. A concerted reaction is one which involves no intermediates during the course of the reaction. Typically, there is a relatively small solvent effect on the rate of reaction, unless the reactants themselves happen to be charged, i.e. carbonium or carbanions.

As used herein, “radiochemical” is intended to encompass any organic, inorganic or organometallic compound comprising a covalently-attached radioactive isotope, any inorganic radioactive ionic solution (e.g., Na[¹⁸F]F ionic solution), or any radioactive gas (e.g., [¹¹C]CO₂), particularly including radioactive molecular imaging probes intended for administration to a patient (e.g., by inhalation, ingestion, or intravenous injection) for tissue imaging purposes, which are also referred to in the art as radiopharmaceuticals, radiotracers, or radioligands. Although the present invention is primarily directed to synthesis of positron-emitting molecular imaging probes for use in PET imaging systems, the invention could be readily adapted for synthesis of any radioactive compound comprising a radionuclide, including radiochemicals useful in other imaging systems, such as single photon emission computed tomography (SPECT).

As used herein, the term “radioactive isotope” refers to isotopes exhibiting radioactive decay (i.e., emitting positrons) and radiolabeling agents comprising a radioactive isotope (e.g., [¹¹C]methane, [¹¹C]carbon monoxide, [¹¹C]carbon dioxide, [¹¹C]phosgene, [¹¹C]urea, [¹¹C]cyanogen bromide, as well as various acid chlorides, carboxylic acids, alcohols, aldehydes, and ketones containing carbon-11). Such isotopes are also referred to in the art as radioisotopes or radionuclides. Radioactive isotopes are named herein using various commonly used combinations of the name or symbol of the element and its mass number (e.g., ¹⁸F, F-18, or fluorine-18). Exemplary radioactive isotopes include I-124, F-18 fluoride, C-11, N-13, and O-15, which have half-lives of 4.2 days, 110 minutes, 20 minutes, 10 minutes, and 2 minutes, respectively. The radioactive isotope is preferably dissolved in an organic solvent, such as a polar aprotic solvent. Preferably, the radioactive isotopes used in the present method include F-18, C-11, I-123, I-124, I-127, I-131, Br-76, Cu-64, Tc-99m, Y-90, Ga-67, Cr-51, Ir-192, Mo-99, Sm-153 and Tl-201. Other radioactive isotopes that may be employed include: As-72, As-74, Br-75, Co-55, Cu-61, Cu-67, Ga-68, Ge-68, I-125, I-132, In-111, Mn-52, Pb-203 and Ru-97.

Optical imaging agent refers to molecules that have wavelength emission greater than 400 nm and below 1200 nm. Examples of optical imaging agents are Alex Fluor, BODIPY, Nile Blue, COB, rhodamine, Oregon green, fluorescein and acridine.

The term “reactive precursor” is directed to any of a variety of molecules that can be chemically modified by addition of an azide or alkynyl group, such as small molecules, natural products, or biomolecules (e.g., peptides or proteins). For ligand formation from two precursor molecules, one of the precursor molecules comprises a non-radioactive isotope of an element having a radioisotope within its nuclide. In certain aspects as used herein, the term “ligand” may refer to the precursor, compounds and imaging probes that bind to the biomacromolecule. The two precursors of the ligand preferably exhibit affinity to separate binding sites (or separate sections of the same binding site or pocket) on a biological target molecule, such as an enzyme. The reactive precursor that has binding affinity for an active site on the biomacromolecule is sometimes referred to herein as the “anchor molecule.” The reactive precursor that has binding affinity for the substrate binding site of a kinase is sometimes referred to herein as the “substrate mimic.” The term “reactive precursor” may also refer to the precursor or compound that are used to prepare the candidate compounds that comprise the library of candidate compounds.

In a particular aspect of the method with the ligand radiochemical embodiment, one of the precursor molecules may also comprise a leaving group that can be readily displaced by nucleophilic substitution in order to covalently attach a radioisotope to the precursor. Exemplary reactive precursors include small molecules bearing structural similarities to existing PET probe molecules, EGF, cancer markers (e.g., p 185HER2 for breast cancer, CEA for ovarian, lung, breast, pancreas, and gastrointestinal tract cancers, and PSCA for prostrate cancer), growth factor receptors (e.g., EGFR and VEGFR), glycoproteins related to autoimmune diseases (e.g., HC gp-39), tumor or inflammation specific glycoprotein receptors (e.g., selectins), integrin specific antibody, virus-related antigens (e.g., HSV glycoprotein D, EV gp), and organ specific gene products.

“Substituted” or a “substituent” as used herein, means that a compound or functional group comprising one or more hydrogen atom of which is substituted by a group (a substituent) such as a —C₁₋₅alkyl, C₂₋₅alkenyl, halogen (chlorine, fluorine, bromine, iodine atom), —CF₃, nitro, amino, oxo, —OH, carboxyl, —COOC₁₋₅alkyl, —OC₁₋₅alkyl, —CONHC₁₋₅alkyl, —NHCOC₁₋₅alkyl, —OSOC₁₋₅alkyl, —SOOC₁₋₅alkyl, —SOONHC₁₋₅alkyl, —NHSO₂C₁₋₅alkyl, aryl, heteroaryl and the like, each of which may be further substituted.

“Substrate mimics” as used herein means compounds that imitate enzyme substrates in their 3-dimensional structures, charge distribution and hydrogen bond donor or acceptor orientation, so they can be recognized by the enzyme active site.

II. Method of Synthesizing Radiochemicals

Traditional ¹⁸F-labeling of small molecules to form PET imaging probes involves displacement of a suitably activated precursor with [18F]fluoride in a compatible reaction media, such as acetonitrile. [18F]fluoride attachment occurs via nucleophilic displacement of substituted sulfonate or nitro moieties, usually at elevated temperatures. Under such reaction conditions, the reactivity of [18F]fluoride may be limited by steric and electronic effects inherent in the target molecule. To complicate matters further, the use of protecting groups may also be needed to enhance the overall yield of the labeled material usually by preventing unwanted side reactions. The selection of protecting groups must be evaluated on a case-by-case basis and their effect, good or otherwise, must be determined experimentally. In order to prepare a large number of [18F]-labeled compounds, every precursor must contain a leaving group as well as optimized protecting groups. Thus, this strategy is not general enough for quickly modifying candidate imaging probes to optimize their physiochemical, pharmacokinetic, and efficacy properties. There is a need in the art for an improved method for quickly synthesizing imaging probes that avoid the problems of the prior art, such as the need for optimized protecting groups. If the assembly of radiolabeled molecules could be accomplished using chemospecific coupling partners under mild conditions, as is the case of click chemistry, there would be an opportunity to prepare diverse radiolabeled molecules for in vivo imaging of many biological targets in a faster and more efficient way than is currently practiced.

The radiochemical synthesis method of the invention utilizes click chemistry to prepare the radioactive ligands that can then be used as PET molecular imaging probes. Click chemistry techniques are described, for example, in the following references, which are incorporated herein by reference in their entirety:
Kolb, H. C.; Finn, M. G.; Sharpless, K. B. Angewandte Chemie, International Edition 2001, 40, 2004-2021.
Kolb, H. C.; Sharpless, K. B. Drug Discovery Today 2003, 8, 1128-1137.
Rostovtsev, V. V.; Green, L. G.; Fokin, V. V.; Sharpless, K. B. Angewandte Chemie, International Edition 2002, 41, 2596-2599.
Tomøe, C. W.; Christensen, C.; Meldal, M. Journal of Organic Chemistry 2002, 67, 3057-3064.
Wang, Q.; Chan, T. R.; Hilgraf, R.; Fokin, V. V.; Sharpless, K. B.; Finn, M. G. Journal of the American Chemical Society 2003, 125, 3192-3193.
Lee, L. V.; Mitchell, M. L.; Huang, S.-J.; Fokin, V. V.; Sharpless, K. B.; Wong, C.-H. Journal of the American Chemical Society 2003, 125, 9588-9589.
Lewis, W. G.; Green, L. G.; Grynszpan, F.; Radic, Z.; Carlier, P. R.; Taylor, P.; Finn, M. G.; Barry, K. Angew. Chem., Int. Ed. 2002, 41, 1053-1057.
Manetsch, R.; Krasinski, A.; Radic, Z.; Raushel, J.; Taylor, P.; Sharpless, K. B.; Kolb, H. C. Journal of the American Chemical Society 2004, 126, 12809-12818.
Mocharla, V. P.; Colasson, B.; Lee, L. V.; Roeper, S.; Sharpless, K. B.; Wong, C.-H.; Kolb, H. C. Angew. Chem. Int. Ed. 2005, 44, 116-120.

Although other click chemistry functional groups can be utilized, such as those described in the above references, the use of cycloaddition reactions is preferred, particularly the reaction of azides with alkynyl groups. In the presence of Cu(I) salts, terminal alkynes and azides undergo 1,3-dipolar cycloaddition forming 1,4-disubstituted 1,2,3-triazoles. In the presence of Ru(II) salts, terminal alkynes and azides undergo 1,3-dipolar cycloaddition forming 1,5-disubstituted 1,2,3-triazoles (Fokin, V. V. et al. Organic Letters 2005, 127, 15998-15999). Alternatively, a 1,5-disubstituted 1,2,3-triazole can be formed using azide and alkynyl reagents (Krasinski, A., Fokin, V. V. & Barry, K. Organic Letters 2004, 1237-1240). Hetero-Diels-Alder reactions or 1,3-dipolar cycloaddition reactions could also be used (see Huisgen 1,3-Dipolar Cycloaddition Chemistry (Vol. 1) (Padwa, A., ed.), pp. 1-176, Wiley; Jorgensen Angew. Chem. Int. Ed. Engl. 2000, 39, 3558-3588; Tietze, L. F. and Kettschau, G. Top. Curr. Chem. 1997, 189, 1-120).

The choice of azides and alkynes as coupling partners is particularly advantageous as they are essentially non-reactive towards each other (in the absence of copper) and are extremely tolerant of other functional groups and reactions conditions. This chemical compatibility helps ensure that many different types of azides and alkynes may be coupled with each other with a minimal amount of side reactions. Radiolabeling processes using such functional groups are general, meaning the [F18]-labeled precursor can include either an alkyne or an azide with no loss of yield or efficiency. Further, labeling conditions are mild, small molecules with many functional groups do not impede labeling, and biomolecules may also undergo labeling. In addition, no protecting groups are required and reaction conditions are suitable for many labeling substrates.

In one aspect, the inventive method involves reaction of a reactive precursor bearing a click chemistry functional group with a radioactive precursor molecule comprising a radioactive isotope covalently attached to a complementary click chemistry functional group (see Reaction 1 and Reaction 2, FIG. 1). The radioactive precursor molecule is preferably a relatively simple molecule that can be formed by nucleophilic substitution of a radioisotope onto a parent molecule comprising the click chemistry functional group covalently attached to a leaving group. For example, the radioactive precursor molecule can comprise a terminal alkynyl group attached to an F-18 atom.

In another aspect, the inventive method involves reaction of a reactive precursor bearing a click chemistry functional group with a radioactive molecule comprising a radioactive isotope and a second reactive precursor attached to both a complementary click chemistry functional group and a leaving group suitable for displacement by a radioactive isotope (see Reaction 3). For example, the radioactive precursor molecule can comprise a terminal alkynyl group attached to an F-18 atom.

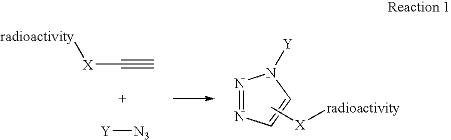

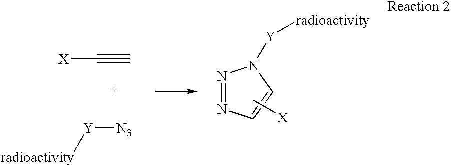

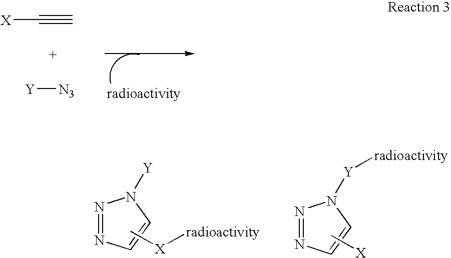

FIG. 1: General methods for preparing labeled compounds for molecular imaging

An exemplary reaction scheme (Scheme I) for forming an analog of FLT (2) is shown below, wherein AZT, which contains an azide group, is reacted with a molecule bearing a terminal alkyne attached to F-18, thereby forming a triazole-linked FLT analog (1). The F-18 precursor is formed in a single step by displacing a leaving group (i.e., —OTs) with F-18.

Because of the mild nature of this coupling, all nucleosides and their analogs may be labeled using this chemistry. For example, the azide analog of guanosine may be 18F-labeled with 18F-propargylfluoride to yield the 18F-labeled triazole-bearing guanosine derivative (Scheme I).

A second reaction scheme is shown in the bottom half of Scheme I. The starting nucleoside scaffold may contain an alkyne. The radiolabeled precursor, 18F-fluoroethylazide, is first prepared and then reacted with the alkyne portion of the nucleoside to form a triazole-bearing 18F-labeled nucleoside analog. If the catalyst is changed to a Ru(II) derivative, the 1,5-substituted triazole may be formed.

By varying the location of the azide and/or alkyne on the nucleoside scaffold, a library of 18F-labeled nucleoside analogs is readily available. In the example shown in FIG. 2 below, a library 18F-labeled thymidine analogs may be prepared by starting with the appropriately alkyne or azide bearing thymidine analog and reacting that analog with either 18F-labeled alkynes or alkyl azides. Some examples are also provided herein.

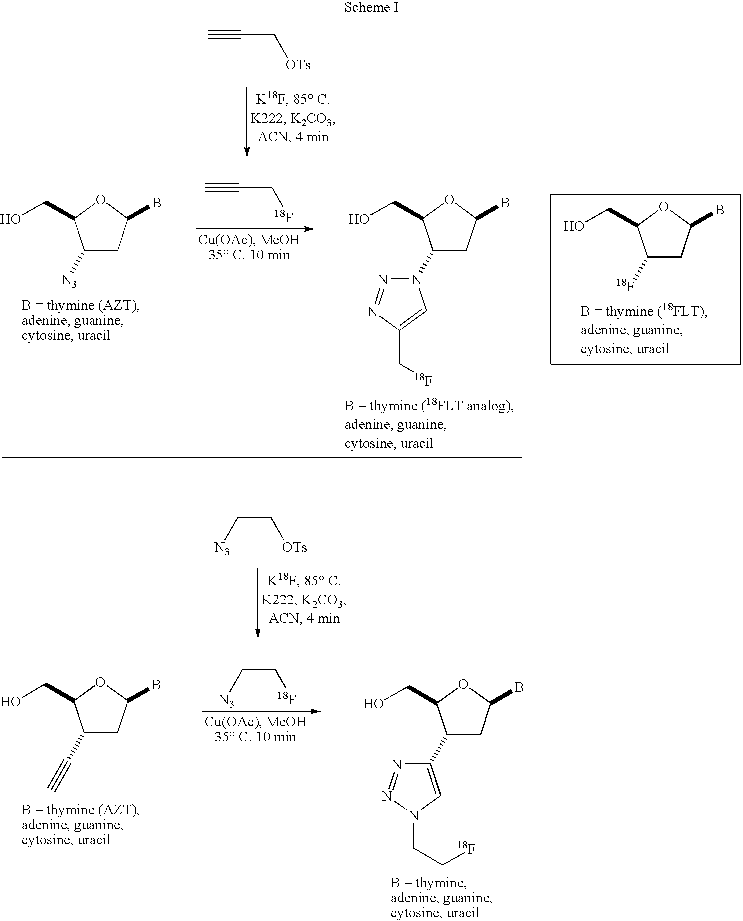

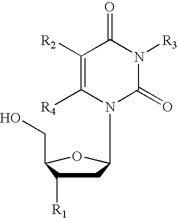

Example 1: R₁ = A—X; R₂ = CH₃; R₃ = H; R₄ = H
Example 2: R₁ = F, OH, H, N₃; R₂ = CH₃; R₃ = X—A;
R₄ = H
Example 3: R₁ = F, OH, H, N₃; R₂ = X—A; R₃ = H; R₄ = H
Example 4: R₁ = F, OH, H, N₃; R₂ = CH₃; R₃ = H;
R₄ = X—A
X = A linker that contains a click chemistry group:

| | |
|---|---|
| 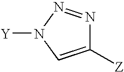 | Y = (CH₂)ₙ, n = 0-3 Z = (CH₂)ₘ, m = 0-3 |

A = A radioisotope for molecular imaging (PET or SPECT).
In case of PET: ¹¹C, ¹⁸F

### EXAMPLES

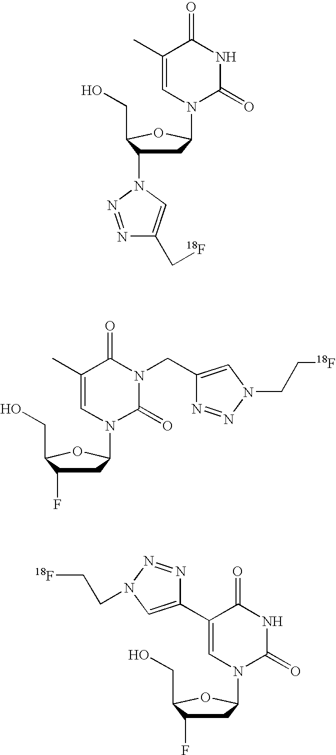

FIG. 2.

Another variation on the labeling theme would be to first react the azide and the alkyne, in this example the alkylazide bears a leaving group, to form triazole followed by displacement of the leaving group with 18F-fluoride (Scheme II).

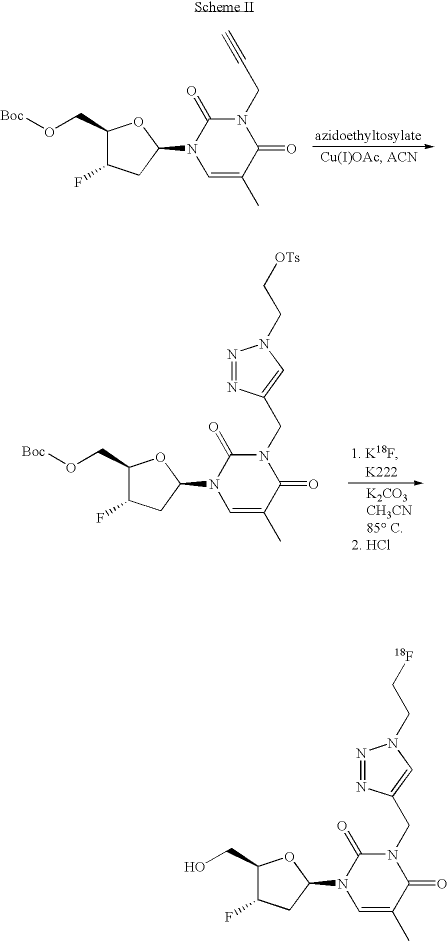

This method of labeling is also ideally suited for labeling of biomacromolecules with radioisotopes. The reactive precursor that is reacted with the radioactive precursor or “tag” can also be any of various disease-related biomolecules, including proteins, carbohydrates, and the like. Any molecule of biological utility that can be chemically modified to include a click chemistry reactive group, such as an azide or an alkynyl group, can be used as the reactive precursor without departing from the present invention. The radioactive precursor is first synthesized and then coupled in aqueous buffer media in the presence of copper (I) salts to afford triazole formation.

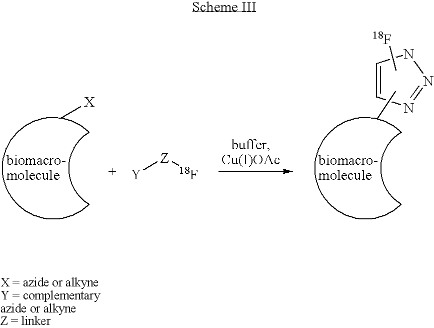

The first reactive precursor is reacted with a solution comprising a radioactive isotope under conditions sufficient to displace the leaving group and covalently attach the radioactive isotope to the first reactive precursor, thereby forming a radioactive reactive precursor. For solutions containing ¹⁸F, the radioactive isotope is typically in the form of a coordination compound consisting of a phase transfer catalyst and salt complex. One common ¹⁸F solution comprises Kryptofix 2.2.2 as the phase transfer catalyst and ¹⁸F in a salt complex with potassium carbonate (K₂CO₃). Both the precursors and the radioisotope solutions are preferably dissolved in a polar aprotic solvent. The polar aprotic solvent used in each reagent can be the same or different, but is typically the same for each reagent. Exemplary polar aprotic solvents include acetonitrile, acetone, 1,4-dioxane, tetrahydrofuran (THF), tetramethylenesulfone (sulfolane), N-methylpyrrolidinone (NMP), dimethoxyethane (DME), dimethylacetamide (DMA), N,N-dimethylformamide (DMF), dimethylsulfoxide (DMSO), and hexamethylphosphoramide (HMPA). Exemplary nucleophilic leaving groups include halogen, pseudohalogen, nitro, diazonium salt and sulfonate ester. Particularly preferred leaving groups include bromine, iodine, tosylate, and triflate.

The radioactive precursor can then be reacted with the second reactive precursor under conditions sufficient to covalently attach the radioactive precursor to the second reactive precursor via a click chemistry reaction between the first and second reactive groups (e.g., between the azide and alkynyl groups), thereby forming the ligand radiochemical. In one variation of the above reaction, methanol is the preferred solvent. However, other polar protic solvents may also be employed, including but not limited to, ethanol, tertiary-butanol, water and buffered mixtures thereof. The ligand radiochemical is then collected and preferably purified, for example, by passing the ligand radiochemical solution through a series of HPLC columns. One column is preferably adapted to remove inorganic impurities (e.g., copper and unreacted F-18) and one column is preferably adapted to remove organic impurities such as Kryptofix.

The solution of radioisotope can be formed using methodology known in the art. For example, in the case of F-18, water collected from a cyclotron containing [¹⁸F]fluoride ion is passed through an anion exchange column in order to trap the F-18 ion. The [¹⁸F]fluoride ion is then released from the resin column using a potassium carbonate aqueous solution, and mixed with a solution of Kryptofix 222 in a polar aprotic solvent such as acetonitrile.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the invention. Although specific terms are employed herein, they are used in a generic and descriptive sense only and not for purposes of limitation.

Aspects of the Invention:

In one embodiment, there is provided a method for preparing a radioactive ligand or radioactive substrate having affinity for a target biomacromolecule, the method comprising:

(a) reacting a first compound comprising i) a first molecular structure; ii) a leaving group; iii) a first functional group capable of participating in a click chemistry reaction; and optionally, iv) a linker between the first functional group and the molecular structure, with a radioactive reagent under conditions sufficient to displace the leaving group with a radioactive component of the radioactive reagent to form a first radioactive compound;

(b) providing a second compound comprising i) a second molecular structure; ii) a second complementary functional group capable of participating in a click chemistry reaction with the first functional group, wherein the second compound optionally comprises a linker between the second compound and the second functional group;

(c) reacting the first functional group of the first radioactive compound with the complementary functional group of the second compound via a click chemistry reaction to form the radioactive ligand or substrate; and

(d) isolating the radioactive ligand or substrate.

In one variation of the above method, the biomacromelecule is selected from the group consisting of enzymes, receptors, DNA, RNA, ion channels and antibodies. In a particular variation, the biomacromolecule is a protein. In certain variation of the method, the target biomacromolecule is a protein that is overexpressed in disease states, such as beta-amyloid in brain tissue of Alzheimer's Disease patients.

According to another variation of the method, the click chemistry reaction is a pericyclic reaction. Preferably, the pericyclic reaction is a cycloaddition reaction. In one variation of the above, the pericyclic reaction is selected from the group consisting of a 1,3-dipolar cycloaddition reaction and a Diels-Alder reaction. In another variation of the method, preferably, the pericyclic reaction is a 1,3-dipolar cycloaddition reaction. In another variation of the method, the click chemistry reaction is a 1,3-dipolar cycloaddition reaction. In one particular variation, the first functional group is an azide and the second functional group is a terminal alkyne, or wherein the first functional group is a terminal alkyne and the second functional group is an azide. In yet another variation, the complementary click functional groups comprises an azide and an alkyne and the click reaction forms the radioactive ligand or substrate comprising a 1,4- or 1,5-disubstituted 1,2,3 triazole. In another variation of the method, the click reaction is performed in the presence of a catalyst, and wherein the catalyst may be a Cu(I) salt or a ruthenium (II) salt.

In a particular preferred variation, the Cu(I) salt is Cu(OAc), and the Ru(II) salt is Cp*RuCl(PPh₃)₂.

The click reaction may also be performed thermally. In one variation, the click reaction is performed at slightly elevated temperatures between 25° C. and 200° C. In one aspect, the reaction may be performed between 25° C. and 150° C., or between 25° C. and 100° C. In another aspect, the click reaction at elevated temperatures may also be performed using a microwave oven. In one variation of the method, the radioactive agent is a coordinating compound comprising a phase transfer catalyst and a salt complex. In another variation, the radioactive agent is selected from the group consisting of n-Bu₄NF-F18, Kryptofix [2,2,2] or potassium carbonate, or potassium bicarbonate, or cesium carbonate, or cesium bicarbonate and/or potassium 18F-fluoride and/or cesium 18F-fluoride.

In a particular variation of the method, the displacement reaction may be performed in a polar aprotic solvent selected from the group consisting of acetonitrile, acetone, 1,4-dioxane, tetrahydrofuran (THF), tetramethylenesulfone (sulfolane), N-methylpyrrolidinone (NMP), dimethoxyethane (DME), dimethylacetamide (DMA), N,N-dimethylformamide (DMF), dimethylsulfoxide (DMSO) and hexamethylphosphoramide (HMPA) and mixtures thereof, and the click reaction is performed in either polar aprotic solvents or in polar protic solvents selected from the group consisting of methanol, ethanol, 2-propanol, tertiary-butanol, n-butanol and/or water or buffered solutions thereof. In a particular variation of the method, the leaving group is selected from the group consisting of halogens, the nitro moiety, diazonium salts and sulfonate esters.

In another variation of the above method, the linker between the first functional group and the first molecular structure or the linker between the second functional group and the second molecular structure, comprises between 1 to 10 atoms in the linker chain. A “linker” as used herein refers to a chain comprising 1 to 10 atoms and may comprise of the atoms or groups, such as C, —NR—, O, S, —S(O)—, —S(O)₂—, CO, —C(NR)— and the like, and wherein R is H or is selected from the group consisting of (C₁₋₁₀)alkyl, (C₃₋₈)cycloalkyl, aryl(C₁₋₅)alkyl, heteroaryl(C₁₋₅)alkyl, amino, aryl, heteroaryl, hydroxy, (C₁₋₁₀)alkoxy, aryloxy, heteroaryloxy, each substituted or unsubstituted. The linker chain may also comprise part of a saturated, unsaturated or aromatic ring, including polycyclic and heteroaromatic rings.

According to a variation of the above method, the first molecular structure or the second molecular structure is a nucleic acid derivative. Also, in certain variations of the method, the nucleic acid derivative is a thymidine derivative. In another variation of the method, the radioactive substrate is prepared according to the process scheme below: 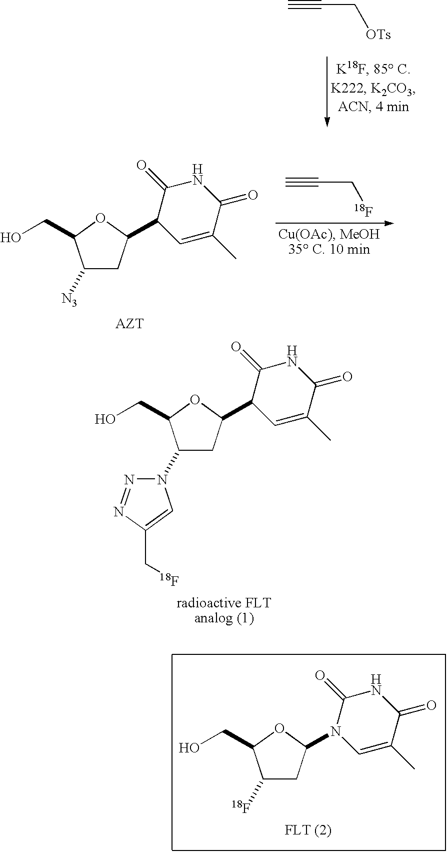
wherein the first molecular structure is des-azido AZT, the first functional group is an azide, the second molecular structure is a —CH₂— group, the leaving group attached to the second molecular structure is —OTs, and the radioactive substrate is the radioactive FLT analog.

In yet another variation of the above method, the radioactive substrate is prepared according to the process scheme below: 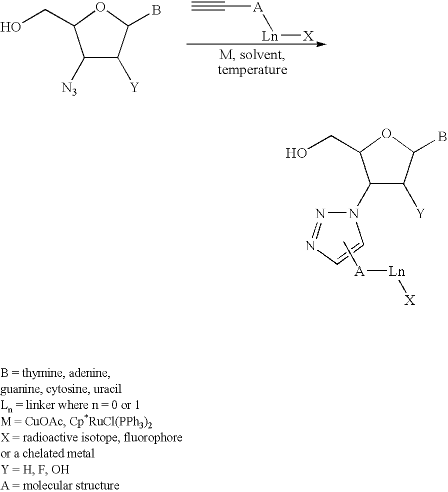
wherein: the base (B) on the ribose ring is selected from the group consisting of adenine, guanine, cytosine, thymine and uracil;

when the catalyst is CuOAc, the reaction forms a 1,4 triazole product or when the catalyst is Cp*RuCl(PPh₃)₂, the reaction forms a 1,5-triazole product;

X is selected from the group consisting of a radioactive isotope, a fluorophore and a chelated metal; and optionally, wherein X is attached to the alkyne via a linker.

According to another embopdiment, there is provided a process for preparing a substrate or ligand according to the process scheme below: 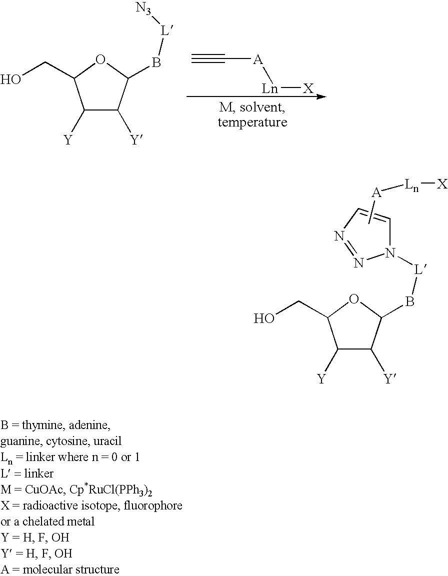
wherein: the base (B) on the ribose ring is selected from the group consisting of adenine, guanine, cytosine, thymine and uracil, and where the base comprises an azide optionally attached to a linker L′, wherein the base are substituted and functionalized as selected from the group consisting of:

1) B=thymine, where the azide is optionally attached via a linker to the 3-position, the 5-methyl or the 6-position;

2) B=cytosine, where the azide is optionally attached via a linker to the 4-N nitrogen, the 5-position or the 6-position;

3) B=uracil, where the azide is optionally attached via a linker to the 3-N nitrogen, the 5-position or the 6-position;

4) B=adenine, where the azide is optionally attached via a linker to the 6-N nitrogen, the 2-position or the 8-position; and

5) B=guanine, where the azide is optionally attached via a linker to the 2-N nitrogen, the 1-N nitrogen or the 8-position;

wherein the catalyst is CuOAc, then the reaction forms a 1,4 triazole or where the catalyst is Cp*RuCl(PPh₃)₂, then the reaction forms a 1,5-triazole; wherein

X is the radioactive element attached to the alkyne via a linker; or

wherein X is a radioactive isotope, fluorophore or chelated metal; and wherein Y is hydrogen, fluorine or hydroxyl.

In particular variations of the method or process, the linker comprises the molecular structure, or wherein the linker and the molecular structure is the same element.

According to another aspect, there is provided a process for preparing a substrate or ligand according to the process below: 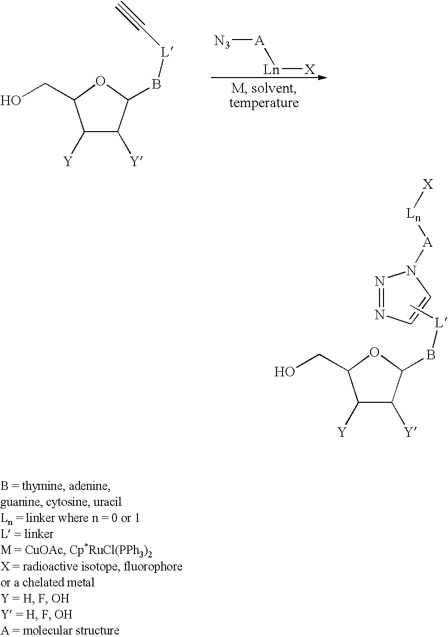
wherein: B is a base attached to the ribose ring and is selected from the group consisting of adenine, guanine, cytosine, thymine and uracil; or

wherein B=thymine and the alkyne is attached optionally via a linker to the 3-position, the 5-methyl, or the 6-position of the ribose; or

wherein B=cytosine and the alkyne is attached optionally via a linker to the 4-N nitrogen, the 5-position or the 6-position; or

wherein B=uracil and the alkyne is attached optionally via a linker to the 3-N nitrogen, the 5-position or the 6-position; or

wherein B=adenine and the alkyne is attached optionally via a linker to the 6-N nitrogen, the 2-position or the 8-position; or

wherein B=guanine and the alkyne is attached optionally via a linker to the 2-N nitrogen, the 1-N nitrogen or the 8-position; and

where the catalyst is CuOAc, the reaction forms a 1,4 triazole, or when the catalyst is Cp*RuCl(PPh₃)₂ the reaction forms a 1,5-triazole; or

wherein X is a radioactive isotope, fluorophore or chelated metal; and Y is hydrogen, fluorine or hydroxyl.

In yet another aspect, there is provided a method for preparing a radioactive ligand or substrate having affinity for a target biomacromolecule, the method comprising:

(a) providing a first compound comprising i) a first molecular structure; ii) a leaving group; iii) a first functional group capable of participating in a click chemistry reaction; and optionally, iv) a linker between the first functional group and the molecular structure;

(b) providing a second compound comprising i) a second molecular structure; ii) a second complementary functional group capable of participating in a click chemistry reaction with the first functional group, wherein the second compound optionally comprises a linker between the second compound and the second functional group;

(c) reacting the first functional group with the complementary functional group of the second compound via a click chemistry reaction to form the ligand or substrate; and

(d) reacting the ligand or substrate with a radioactive reagent under conditions sufficient to displace the leaving group with a radioactive component of the radioactive reagent to form the radioactive ligand or substrate; and

(e) isolating the radioactive ligand or substrate.

In one variation of each of the above method, the biomacromelecule is selected from the group consisting of enzymes, receptors, DNA, RNA, ion channels and antibodies. In another variation of each of the above methods, the biomacromolecule is a protein. In yet another variation of each of the above method, the click chemistry reaction is a pericyclic reaction, and in certain variations, the pericyclic reaction is a cycloaddition reaction. In particular variation of each of the above, the pericyclic reaction is selected from the group consisting of a 1,3-dipolar cycloaddition reaction and a Diels-Alder reaction. In a particular preferred variation of the above method, the pericyclic reaction is a 1,3-dipolar cycloaddition reaction.

In one variation of the above method, the first functional group is an azide and the second functional group is an alkyne, or wherein the first functional group is an alkyne and the second functional group is an azide. According to the above variations of the method, the complementary click functional groups comprises an azide and an alkyne and the click reaction forms the radioactive ligand or substrate comprising a 1,4- or 1,5-disubstituted 1,2,3 triazole. In a particular variation, the click reaction is performed in the presence of a catalyst, and the catalyst is a Cu(I) salt or a ruthenium (II) salt. In a particular preferred variation, the Cu(I) salt is Cu(OAc). In a particular variation, the Ru(II) salt is Cp*RuCl(PPh₃)₂.

In certain procedures of the above method, the reaction may be performed at elevated temperatures. In one variation, the click reaction is performed at slightly elevated temperatures between 25° C. and 200° C. In particular variations of the method, the radioactive agent is a coordinating compound comprising a phase transfer catalyst and a salt complex. In yet another variation, the radioactive agent is selected from the group consisting of n-Bu₄NF-F18, Kryptofix [2,2,2] and potassium carbonate, potassium bicarbonate, cesium carbonate, cesium bicarbonate and/or potassium 18F-fluoride.

According to another variation, there is provided a method for preparing a labeled biomacromolecule, the method comprising:

(a) reacting a first compound comprising i) a first molecular structure; ii) a leaving group; iii) a first functional group capable of participating in a click chemistry reaction; and optionally, iv) a linker between the first functional group and the molecular structure, with a radioactive reagent under conditions sufficient to displace the leaving group with a radioactive component of the radioactive reagent to form a first radioactive compound;

(b) providing a second compound comprising i) a macromolecule; ii) a second complementary functional group capable of participating in a click chemistry reaction with the first functional group, wherein the biomacromolecule optionally comprises a linker between the biomacromolecule and the second functional group;

(c) reacting the first functional group of the first radioactive compound with the complementary functional group of the biomacromolecule via a click chemistry reaction to form the radioactive biomacromolecule; and

(d) isolating the radioactive biomacromolecule.

In a variation of the above method, the biomacromelecule is selected from the group consisting of enzymes, receptors, DNA, RNA, ion channels and antibodies. In another variation, the biomacromolecule is a protein. In yet another variation of the above method, the protein is epidermal growth factor (EGF).

In another aspect, there is provided a method for preparing a radioactive ligand or substrate, the method comprising:

(a) providing a first compound comprising i) a first molecular structure; ii) a leaving group; iii) a first functional group capable of participating in a click chemistry reaction; and optionally, iv) a linker between the first functional group and the molecular structure;

(b) providing a second compound comprising i) a biomacromolecule; ii) a second complementary functional group capable of participating in a click chemistry reaction with the first functional group, wherein the second compound optionally comprises a linker between the biomacromolecule and the second functional group;

(c) reacting the first functional group with the complementary functional group of the second compound via a click chemistry reaction to form the ligand or substrate; and

(d) reacting the ligand or substrate with a radioactive reagent under conditions sufficient to displace the leaving group with a radioactive component of the radioactive reagent to form the radioactive ligand or substrate; and

(e) isolating the radioactive ligand or substrate.

According to one variation of each of the above method, the biomacromelecule is selected from the group consisting of enzymes, receptors, DNA, RNA, ion channels and antibodies. According to another variation, the biomacromolecule is a protein. According to yet another variation, the leaving group is selected from the group consisting of halogens, the nitro moiety, diazonium salts and sulfonate esters.

In each of the above aspects of the disclosure as recited herein, including all aspects, embodiments and variations and representative examples, are intended to be interchangeable where applicable, such that the various aspects, embodiments and variations may be combined interchangeably and in different permutations. For example, a particular first molecular structure comprising a first functional group without a linker may undergo a 1,3-dipolar cycloaddition reaction with a second molecular structure with a complementary functional group without a linker, or alternatively, the same first molecular structure comprising the functional group with a linker may undergo a 1,3-dipolar cycloaddition reaction with a second molecular structure comprising a complementary functional group comprising a linker between the molecular structure and the complementary functional group. These and other permutations and variations are intended to be included in the aspects of the invention.

### EXAMPLE

### Synthesis of 3′-deoxy-3′-[(4-[¹⁸F]fluoromethyl)-[1,2,3]triazole]thymidine

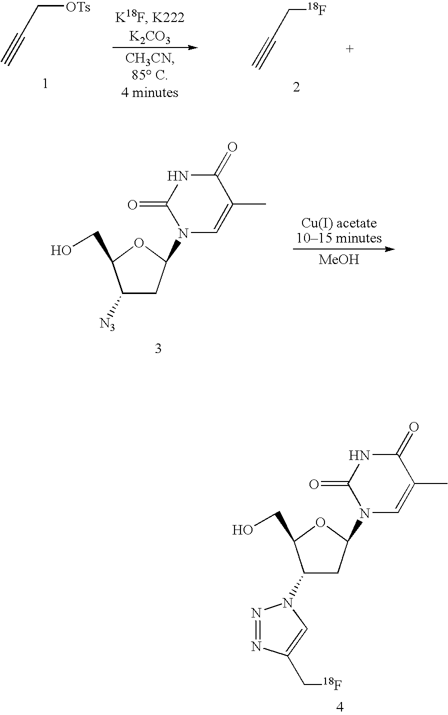 Click In-Situ 2-Step F-18 3′-Triazole Experimental

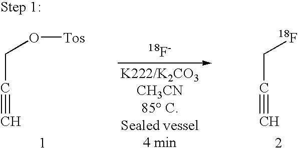

Oxygen-18 water (>97% enriched) was irradiated using 11 MeV protons (RDS-111 Eclipse, Siemens Molecular Imaging) to generate [¹⁸F]fluoride ion in the usual way. At the end of the bombardment, the [¹⁸O]water containing [¹⁸F]fluoride ion was transferred from the tantalum target to an automated nucleophilic fluorination module (explora RN, Siemens Biomarker Solutions). Under computer control, the [¹⁸O]water/[¹⁸F]fluoride ion solution was transferred to a small anion exchange resin column (Chromafix 45-PS-HCO3, Machery-Nagel) which had previously been rinsed with water (5 mL), aqueous potassium bicarbonate (0.5 M, 5 mL), and water (5 mL). The [¹⁸O]water (1.8 mL) was recovered for subsequent purification and reuse. The trapped [¹⁸F]fluoride ion was eluted into the reaction vessel with a solution of potassium carbonate (3.0 mg) in water (0.4 mL). A solution of Kryptofix 222 (K222, 20 mg) in acetonitrile (1.0 mL) was added, and the mixture was heated (70 to 95° C.) under vacuum and a stream of argon to evaporate the acetonitrile and water. After cooling, to the residue of “dry” reactive [¹⁸F]fluoride ion, K222, and potassium carbonate, was added a solution of propargyl tosylate (1, 10.0 mg, 47.6 μmol) in acetonitrile (0.8 mL). The reaction mixture was heated to 85° C. in a sealed vessel (Pₘₐₓ=1.8 bar) for 4 minutes with stirring (magnetic). The mixture was then cooled to 35° C.

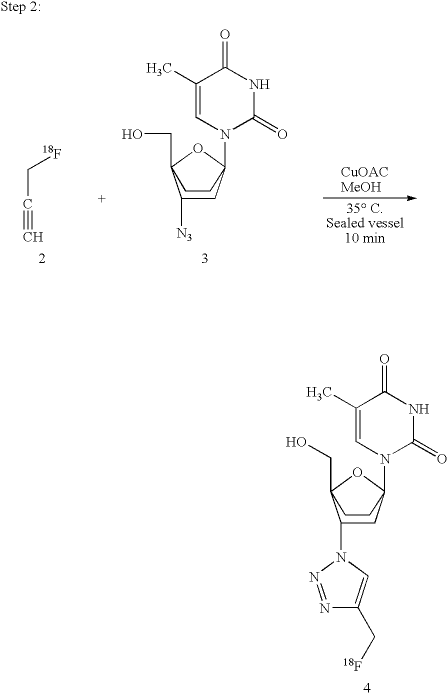

To the reaction mixture containing 2 was added a solution of 3′-deoxy-3′-azidothymidine (AZT, 3, 13 mg, 48.7 μmol) and copper(I) acetate (12 mg, 98 μmol) in methanol (0.5 mL), and the mixture was stirred (magnetic) in a sealed vessel at 35° C. for 10 minutes.

In order to hydrolyze any residual tosylate, aqueous hydrochloric acid (1.0 M, 1.0 mL), was added and the mixture was heated to 105° C. for 3 minutes. After cooling to 35° C., aqueous sodium acetate (2.0 M, 0.5 mL) was added with stirring. The reaction mixture was transferred to a sample loop (1.5 mL), and injected onto a semi-prep HPLC column (Phenomenex Gemini 5μ C18, 250×10 mm, 8% ethanol, 92% 21 mM phosphate buffer pH 8.0 mobile phase, 6.0 mL/min). The product 3′-deoxy-3′-[(4-[¹⁸F]fluoromethyl)-[1,2,3]triazole]thymidine (4, [¹⁸F]FMTT) eluted at 16-18 minutes as monitored by flow-through radiation detection and UV (254 nm). The HPLC eluate containing the product (10-12 mL) was passed through a 0.22 μm sterile filter into a sterile vial.

A typical production run starting with about 500 mCi of [¹⁸F]fluoride ion gave 14.2 mCi (20.7 mCi at EOB, 4.1% yield) of isolated product after 60 minutes of synthesis and HPLC purification.

The collected product was analyzed by HPLC (Phenomenex Gemini 5μ C18, 150×4.6 mm, 12% ethanol, 88% water mobile phase, 1.0 mL/min). As monitored by radioactivity and UV (267 nm) detection, this product had a retention time of 5 minutes and a radiochemical purity of >96.0%.
Synthesis of Triazole Precursor and Standard:

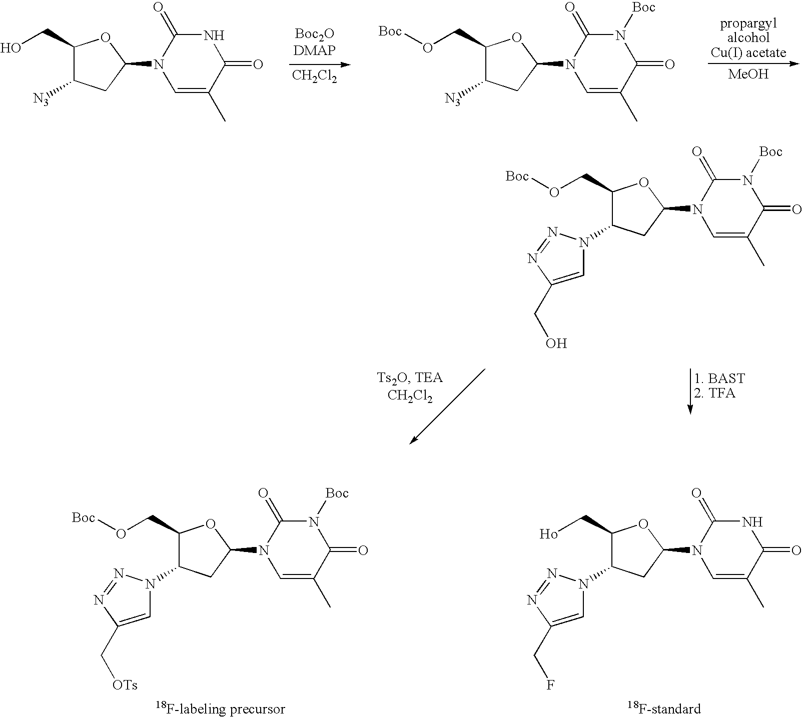

### Synthesis of 3-N-5′-O-BisBoc AZT

To a round bottom flask containing AZT (3.2 g, 11.99 mmol), DMAP (8.1 g, 71.91 mmol) and CH₂Cl₂ (20 mL) was added Boc₂O (15.7 g, 71.91 mmol) with venting. The reaction quickly became yellow. The reaction was stirred overnight at room temperature. The reaction was then poured onto water and extracted into CH₂Cl₂. The combined organics were washed with water, dried (MgSO₄), filtered and concentrated to dryness. The crude material was purified on silica gel using CH₂Cl₂ as the eluent to afford 5 g (89.3%) of a white solid.

¹H NMR (300 MHz, CDCl₃) δ: 1.50 (9H, s), 1.61 (9H, s), 1.95 (3H, d, J=3.0 Hz); 2.39-2.48 (2H, m), 4.05-4.07 (1H, m), 4.23-4.25 (1H, m), 4.32-4.34 (2H, m), 6.20 (1H, t, J=6.0 Hz), 7.46 (1H, s).

MS (electrospray): 490 (M+23)

### Synthesis of 3-N-5′-O-BisBoc-3′-[4-hydroxymethyl-1,2,3-triazole]thymidine

To a round bottom flask containing the azide (1.4 g, 3 mmol), propargyl alcohol (201 mg, 3.6 mmol) and MeOH (6 mL) was added Cu(I)acetate (142 mg, 1.2 mmol). TLC (Et₂O) indicated ˜80% consumption of starting material after 1 minute and ˜100% consumption of starting material after 4 minutes. Water was added to the reaction which generated a ppt. The ppt was isolated via filtration. The crude material was then purified on silica.

¹H NMR (300 MHz, CDCl₃) δ: 1.50 (9H, s), 1.61 (9H, s), 1.98 (3H, s,); 2.71-2.81 (1H, m), 3.02-3.11 (1H, m), 4.38 (2H, dq, J=12, 3 Hz), 4.63-4.67 (1H, m), 4.82 (2H, s), 5.20-5.28 (2H, m), 6.36 (1H, dd, J=9.0, 6.0 Hz), 7.50 (1H, d, J=3.0 Hz), 7.64 (1H, s)

¹³C NMR (75 MHz, CDCl₃) δ: 12.69, 27.42, 27.73, 38.42, 56.35, 59.15, 65.06, 82.12, 83.53, 86.17, 87.01, 111.00, 121.66, 135.10, 147.76, 148.34, 152.78, 161.19

MS (electrospray): 524 (M+H), 546 (M+23)

### Synthesis of 3-N-5′-O-BisBoc-3′-[4-O-tosylmethyl-1,2-3-triazole]thymidine

To a round bottom flask containing triazole (102 mg, 0.2 mmol), TEA (270 μL, 1.95 mmol), DMAP (2 mg, 0.02 mmol) and CH₂Cl₂ (5 mL) at −20° C. was added Ts₂O (152 mg, 0.8 mmol). The reaction stirred at −20° C. for 3 hrs. TLC (EtOAc) indicated that all starting material was consumed. The reaction was then concentrated to dryness and the residue was purified on silica gel using 40% EtOAc:Hex as the eluent to afford 91 mg (68.9%) of a white solid.

¹H NMR (300 MHz, CDCl₃) δ: 1.50 (9H, s), 1.61 (9H, s), 1.98 (3H, s,); 2.47 (3H, s), 2.71-2.81 (1H, m), 3.02-3.11 (1H, m), 4.38 (2H, dq, J=12, 3 Hz), 4.56-4.61 (1H, m), 5.19 (2H, s), 5.20-5.28 (2H, m), 6.36 (1H, dd, J=9.0, 6.0 Hz), 7.35 (1H, s), 7.38 (1H, s), 7.50 (1H, d, J=3.0 Hz), 7.64 (1H, s), 7.77 (1H, s), 7.78 (1H, s), 7.82 (1H, s).

¹³C NMR (75 MHz, CDCl₃) δ: 12.67, 21.67, 27.42, 27.72, 38.38, 59.34, 62.86, 64.99, 82.03, 83.51, 86.21, 86.95, 110.98, 127.99, 135.41, 145.28, 147.75, 148.29, 152.75, 161.17.

### Synthesis of 3-N-5′-O-BisBoc-3′-[4-fluoromethyl-1,2,3-triazole]thymidine

To a round bottom flask containing the starting alcohol (105 mg, 0.2 mmol) and CH₂Cl₂ (5 mL) at 0° C. was added BAST (44 mg, 0.2 mmol). The reaction was stirred for 2 hrs. TLC (1:1 EtOAc:Hex) indicated almost complete consumption of starting material. The reaction was poured onto sat'd NaHCO₃ and extracted into CH₂Cl₂. The combined organics were dried (MgSO4), filtered, concentrated to dryness and purified on silica gel using 1:1 EtOAc:Hex as the eluent to afford 58 mg (55%) of a white solid.

MS (electrospray): 526 (M+H), 548 (M+23)

¹H NMR (300 MHz, CDCl₃) δ: 1.50 (9H, s), 1.61 (9H, s), 1.98 (3H, s,); 2.75-2.84 (1H, m), 3.05-3.15 (1H, m), 4.38 (2H, dq, J=12, 3 Hz), 4.63-4.67 (1H, m), 5.23-5.28 (2H, m), 5.51 (2H, d, J=51 Hz), 6.36 (1H, dd, J=9.0, 6.0 Hz), 7.50 (1H, d, J=3.0 Hz), 7.77 (1H, s).

### Synthesis of 3′-[4-fluoromethyl-1,2,3-triazole]thymidine

To a round bottom flask containing fluorotriazole (52 mg, 0.1 mmol) was added TFA (1 mL). The reaction stirred at RT for 1 hr. The reaction was then concentrated to dryness in vacuo and purified on silica gel using 10% MeOH:CH₂Cl₂ as the eluent to afford 10 mg (32.5%) of a clear colorless oil.

MS (electrospray): 326 (M+H), 348 (M+23)

¹H NMR (300 MHz, CDCl₃) δ: 1.96 (3H, s,); 2.89-2.98 (1H, m), 3.03-3.12 (1H, m), 3.78 (1H, dd, J=6.0, 3.0 Hz), 4.04 (1H, dd, J=6.0, 3.0 Hz), 4.44-4.48 (1H, m), 5.45-5.53 (2H, m), 5.52 (2H, d, J=48 Hz), 6.18 (1H, t, J=9.0, 6.0 Hz), 7.34 (1H, s), 7.78 (1H, d, J=3.0 Hz), 8.33 (1H, br s).

¹⁹F NMR (282 MHz, CDCl₃) δ: −208.1087
Click F-18 3′-Triazole Experimental

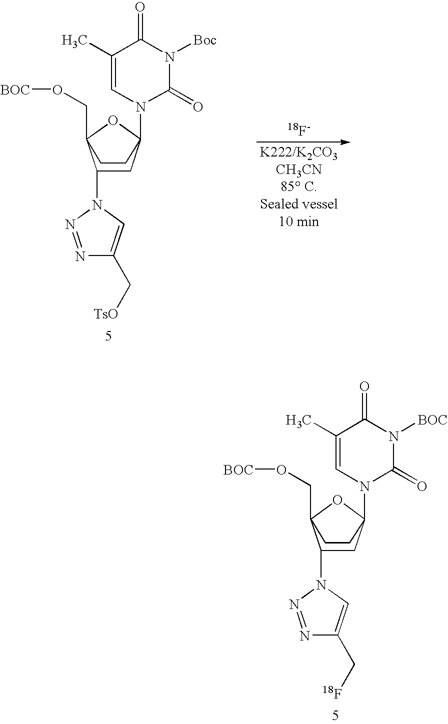

Oxygen-18 water (>97% enriched) was irradiated using 11 MeV protons (RDS-111 Eclipse, Siemens Molecular Imaging) to generate [¹⁸F]fluoride ion in the usual way. At the end of the bombardment, the [¹⁸O]water containing [¹⁸F]fluoride ion was transferred from the tantalum target to an automated nucleophilic fluorination module (explora RN, Siemens Biomarker Solutions). Under computer control, the [¹⁸O]water/[¹⁸F]fluoride ion solution was transferred to a small anion exchange resin column (Chromafix 45-PS-HCO3, Machery-Nagel) which had previously been rinsed with water (5 mL), aqueous potassium bicarbonate (0.5 M, 5 mL), and water (5 mL). The [¹⁸O]water (1.8 mL) was recovered for subsequent purification and reuse. The trapped [¹⁸F]fluoride ion was eluted into the reaction vessel with a solution of potassium carbonate (3.0 mg) in water (0.4 mL). A solution of Kryptofix 222 (K222, 20 mg) in acetonitrile (1.0 mL) was added, and the mixture was heated (70 to 95° C.) under vacuum and a stream of argon to evaporate the acetonitrile and water. After cooling, to the residue of “dry” reactive [¹⁸F]fluoride ion, K222, and potassium carbonate, was added a solution of 3′-deoxy-3′-[(4-p-toluenesulfonyloxy)methyl)-5′-O-Boc-3-N-Boc-[1,2,3]triazole]thymidine (“3′-triazole-thymidine-tosylate”) (5, 26.7 mg, 39.4 μmol) in acetonitrile (0.9 mL). The reaction mixture was heated to 85° C. in a sealed vessel (Pₘₐₓ=2.1 bar) for 10 minutes with stirring (magnetic). The mixture was cooled to 55° C. and most of the acetonitrile was evaporated under vacuum and a stream of argon as before.

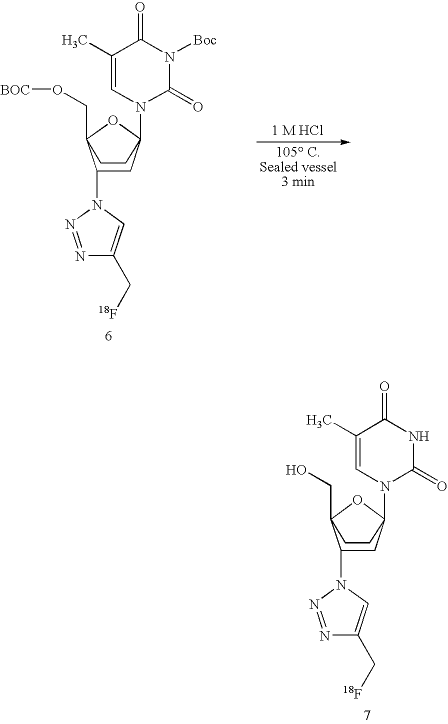

To the crude protected [¹⁸F]fluorinated intermediate (6) was added aqueous hydrochloric acid (1.0 M, 1.0 mL), and the mixture was heated to 105° C. for 3 minutes. After cooling to 35° C., aqueous sodium acetate (2.0 M, 0.5 mL) was added with stirring. The reaction mixture was transferred to a sample loop (1.5 mL), and injected onto a semi-prep HPLC column (Phenomenex Gemini 5μ C18, 250×10 mm, 8% ethanol, 92% 21 mM phosphate buffer pH 8.0 mobile phase, 5.0 mL/min). The product 3′-deoxy-3′-[(4-[¹⁸F]fluoromethyl)-[1,2,3]triazole]thymidine (7, [¹⁸F]FMTT) eluted at 15-18 minutes as monitored by flow-through radiation detection and UV (254 nm). The HPLC eluate containing the product (14-16 mL) was passed through a 0.22 μm sterile filter into a sterile vial.

A typical production run starting with about 800 mCi of [¹⁸F]fluoride ion gave 404 mCi (557 mCi at EOB, 69% yield) of isolated product after 51 minutes of synthesis and HPLC purification.

The collected product was analyzed by HPLC (Phenomenex Gemini 5μ C18, 150×4.6 mm, 12% ethanol, 88% water mobile phase, 1.0 mL/min). As monitored by radioactivity and UV (267 nm) detection, this product had a retention time of 8 minutes and a radiochemical purity of >99.0%.
Synthesis of 3N-triazole Precursor and Standard:

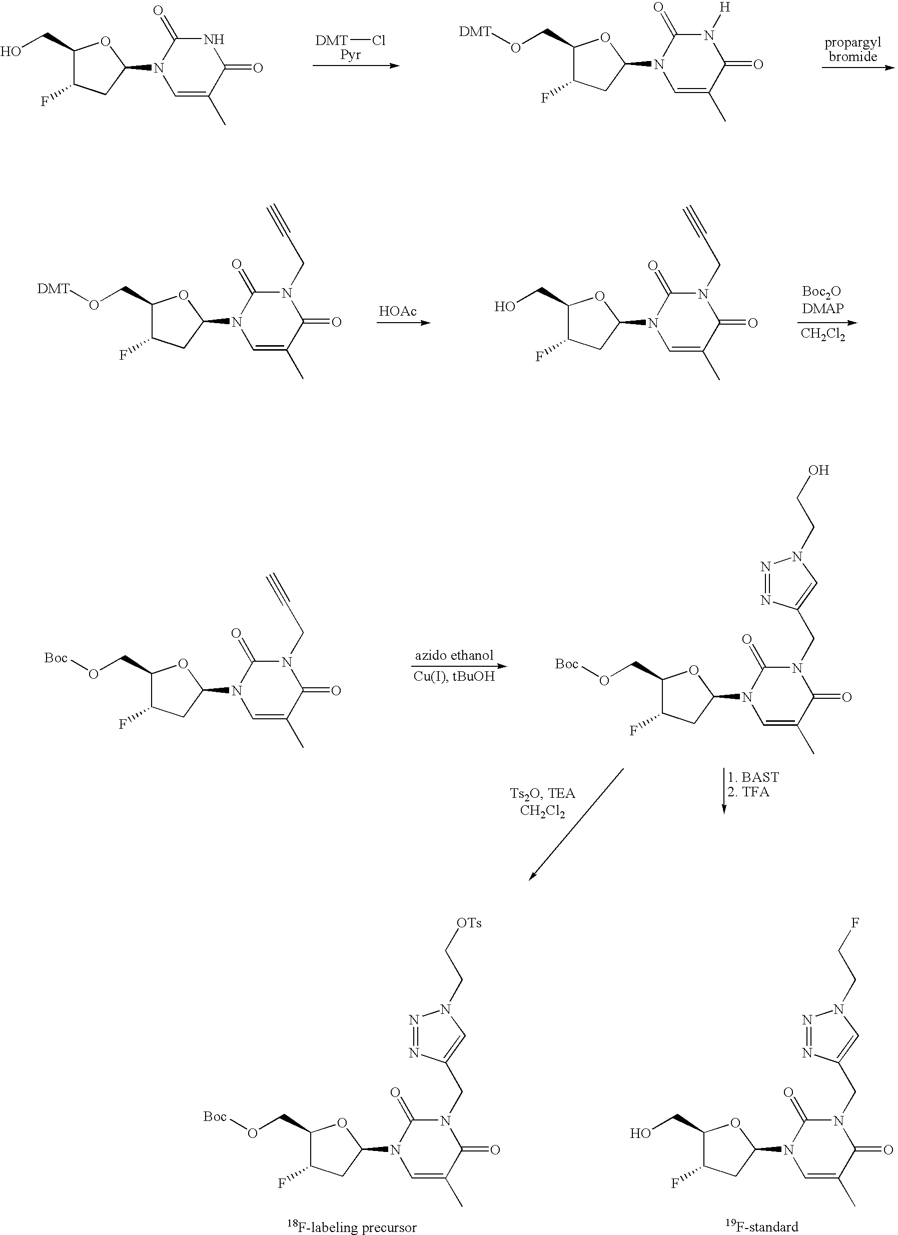

### Synthesis of 5′-O-DMT FLT

To a round bottom flask containing FLT (244 mg, 1 mmol) and TEA (700 uL, 5 mmol) was added DMT-Cl (509 mg, 1.5 mmol). The reaction was stirred overnight. The reaction was then poured onto water and extracted into CH₂Cl₂. The combined organics were dried (MgSO₄), filtered and concentrated to dryness. All was carried on to the next step.

### Synthesis of 3-N-propargyl-5′-O-DMT FLT

To a round bottom flask containing DMT-FLT (546 mg, 1 mmol), DMF (10 mL) and K₂CO₃ (1 g) was added propargyl bromide (179 mg, 1.2 mmol). The reaction was stirred at RT for 3 hrs. TLC (1:1 Et₂O:Hex) indicated complete consumption of starting material. The reaction was poured onto water and extracted into CH₂Cl₂. The combined organics were washed with water (10×'s), dried (MgSO₄), filtered and concentrated to dryness. All was carried on to the next step.

### Synthesis of 3-N-propargyl FLT

To a round bottom flask containing DMT-propargyl FLT (584 mg, 1 mmol) was added HOAc (10 mL). The reaction was heated at reflux for 3 hours. TLC (40% EtOAc:Hex) indicated that the reaction never went to completion. The reaction was then concentrated in vacuo and the residue was purified on silica gel using CH₂Cl₂ to first load the sample followed by 40% EtOAc:Hex to afford 146 mg (52%) of a clear colorless oil.

### Synthesis of 3-N-propargyl-5′-O-Boc FLT

To a round bottom flask containing propargyl FLT (146 mg, 0.52 mmol), DMAP (3 mg, 0.025 mmol), TEA (144 uL, 1.04 mmol) and CH₂Cl₂ (5 mL) was added Boc₂O (136 mg, 0.62 mmol) with venting. The reaction quickly became yellow. The reaction was stirred for 1 hr at room temperature. TLC (50% EtOAc:Hex) indicated complete consumption of starting material. The reaction was then poured onto water and extracted into CH₂Cl₂. The combined organics were washed with water, dried (MgSO₄), filtered and concentrated to dryness. All was carried on to the next step.

### Synthesis of 3-N-(1-hydroxyethyl-4-methylene)-5′-O-Boc-3′-deoxy-3′-fluoro thymidine

To a round bottom flask containing Boc-propargyl FLT (198 mg, 0.51 mmol), azidoethanol (25% pure, 271 mg, 0.78 mmol), sodium ascorbate solution (0.1M, 778 uL), tBuOH (2 mL) and water (2 mL) was added CuSO₄ solution (0.04 M, 972 uL). The reaction went from yellow to brown to yellow all within 1 minute. The reaction was stirred overnight. The reaction was then poured onto sat'd NaHCO₃ and extracted into EtOAc. The combined organics were dried (MgSO₄), filtered and concentrated to dryness. The residue was purified on silica gel using EtOAc (to remove a yellow-colored by product) followed by 10% MeOH:CH₂Cl₂ to afford 157 mg (65.6%) of a white solid.

### Synthesis of 3-N-(1-O-Tosylethyl-4-methylene)-5′-O-Boc-3′-deoxy-3′-fluoro thymidine

To a round bottom flask containing the alcohol (106 mg, 0.23 mmol), CH₂Cl₂ (5 mL), DMAP (3 mg, 0.02 mol), and TEA (315 uL, 2.26 mmol) at −20° C. was added Ts₂O (172 mg, 0.9 mmol). The reaction stirred for 3 hrs. The reaction was then poured onto water and extracted into CH₂Cl₂. The combined organics were dried (MgSO₄), filtered and concentrated to dryness. The residue was purified on silica gel using CH₂Cl₂ to load the material followed by elution with EtOAc to afford 120 mg (83.7%) of a white solid.

### Synthesis of 3-N-(1-fluoroethyl-4-methylene)-5′-O-Boc-3′-deoxy-3′-fluoro thymidine

To a round bottom flask at −78° C. containing the alcohol (46 mg, 0.1 mmol) in CH₂Cl₂ (2 mL) was added BAST (43 μL, 0.2 mmol). The reaction was stirred for 30 min, and then warmed up to RT for 30 min. The reaction was then poured onto sat'd NaHCO₃ and extracted into CH₂Cl₂. The combined organics were dried (MgSO₄), filtered and concentrated to dryness. All was carried on to the next step.

### Synthesis of 3-N-(1-fluoroethyl-4-methylene)-3′-deoxy-3′-fluoro thymidine

To a round bottom flask containing the fluoro compound (47 mg. 0.1 mmol) was added TFA (1 mL). The reaction was stirred at RT for 3 hrs. The reaction was then concentrated to dryness and the residue was purified on silica gel using 2.5% MeOH:CH₂Cl₂ to afford 12 mg (32.3%) of a white solid.
Click F-18 3-N-Triazole Experimental

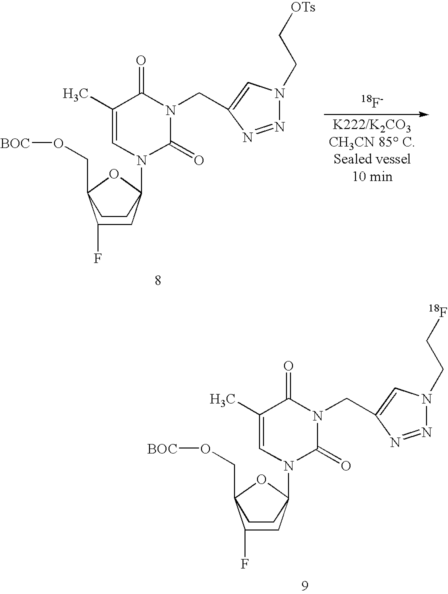

Oxygen-18 water (>97% enriched) was irradiated using 11 MeV protons (RDS-111 Eclipse, Siemens Molecular Imaging) to generate [¹⁸F]fluoride ion in the usual way. At the end of the bombardment, the [¹⁸O]water containing [¹⁸F]fluoride ion was transferred from the tantalum target to an automated nucleophilic fluorination module (explora RN, Siemens Biomarker Solutions). Under computer control, the [¹⁸O]water/[¹⁸F]fluoride ion solution was transferred to a small anion exchange resin column (Chromafix 45-PS-HCO3, Machery-Nagel) which had previously been rinsed with water (5 mL), aqueous potassium bicarbonate (0.5 M, 5 mL), and water (5 mL). The [¹⁸O]water (1.8 mL) was recovered for subsequent purification and reuse. The trapped [¹⁸F]fluoride ion was eluted into the reaction vessel with a solution of potassium carbonate (3.0 mg) in water (0.4 mL). A solution of Kryptofix 222 (K222, 20 mg) in acetonitrile (1.0 mL) was added, and the mixture was heated (70 to 95° C.) under vacuum and a stream of argon to evaporate the acetonitrile and water. After cooling, to the residue of “dry” reactive [¹⁸F]fluoride ion, K222, and potassium carbonate, was added a solution of 3-N-[1-(2′-p-toluenesulfonyloxy)ethyl)-1H-[1,2,3]triazol-4-ylmethyl]-3′-deoxy-3′-fluoro-5′-Boc-thymidine (“3-N-triazole-thymidine-tosylate”) (8, 20.0 mg, 32.1 μmol) in acetonitrile (0.9 mL). The reaction mixture was heated to 85° C. in a sealed vessel (Pₘₐₓ=2.1 bar) for 10 minutes with stirring (magnetic). The mixture was cooled to 55° C. and most of the acetonitrile was evaporated under vacuum and a stream of argon as before.

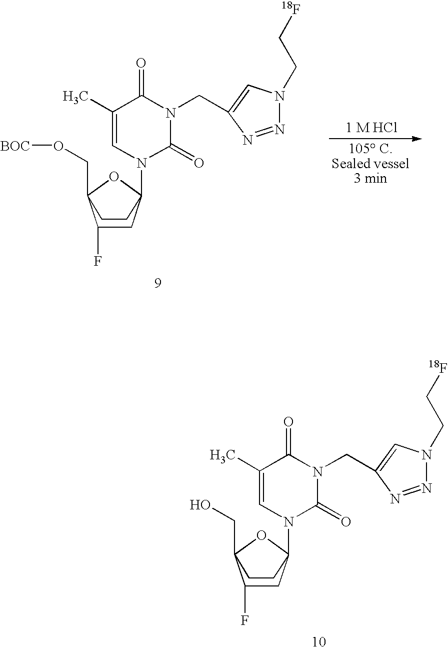

To the crude protected [¹⁸F]fluorinated intermediate (9) was added aqueous hydrochloric acid (1.0 M, 1.0 mL), and the mixture was heated to 105° C. for 3 minutes. After cooling to 35° C., aqueous sodium acetate (2.0 M, 0.5 mL) was added with stirring. The reaction mixture was transferred to a sample loop (1.5 mL), and injected onto a semi-prep HPLC column (Phenomenex Gemini 5μ C18, 250×10 mm, 8% ethanol, 92% 21 mM phosphate buffer pH 8.0 mobile phase, 6.0 mL/min). The product 3-N-[1-(2′-[¹⁸F[fluoroethyl)-1H-[1,2,3]triazol-4-yl-methyl]-3′-deoxy-3′-fluorothymidine (10, [¹⁸F]FETFLT) eluted at 28-29 minutes as monitored by flow-through radiation detection and UV (254 nm). The HPLC eluate containing the product (10-12 mL) was passed through a 0.22 μm sterile filter into a sterile vial.

A typical production run starting with about 475 mCi of [¹⁸F]fluoride ion gave 299 mCi (439 mCi at EOB, 92% yield) of isolated product after 61 minutes of synthesis and HPLC purification.

The collected product was analyzed by HPLC (Phenomenex Gemini 5μ C18, 150×4.6 mm, 20% ethanol, 80% water mobile phase, 1.0 mL/min). As monitored by radioactivity and UV (267 nm) detection, this product had a retention time of 6.5 minutes and a radiochemical purity of >99.0%.
Base-Modified FLT Analog 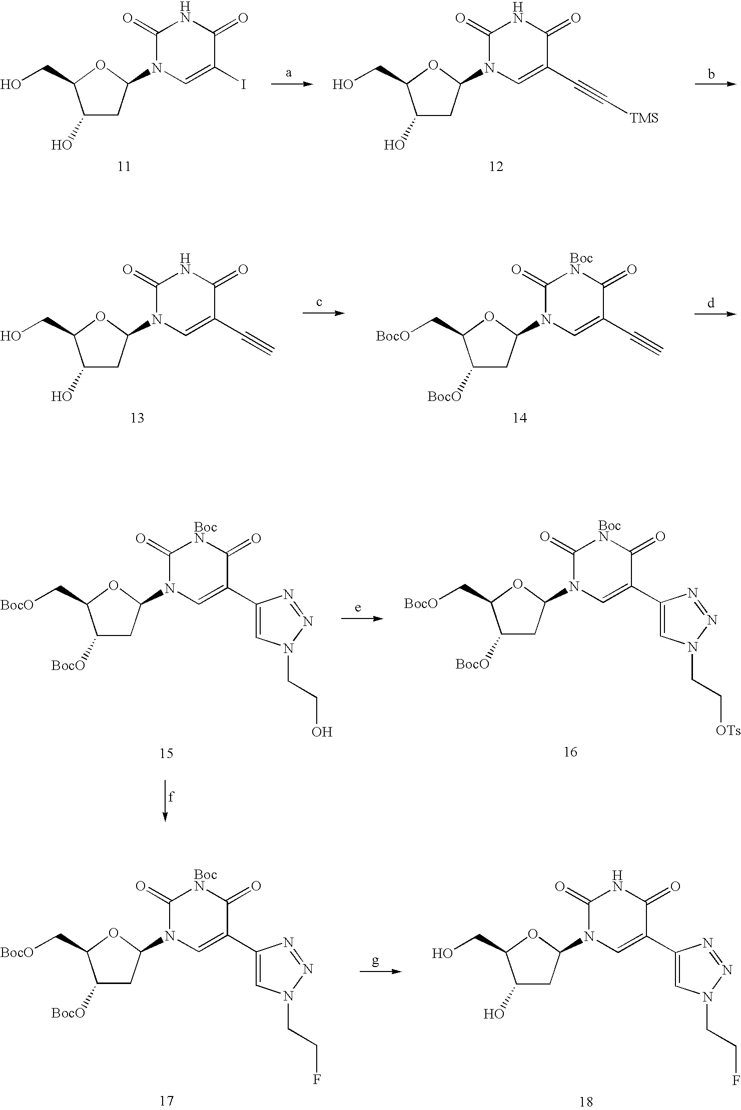
FIG. 3. Synthesis of thymine-modified analog 18. Reagents and conditions: (a) ethynyltrimethylsilane, (Ph₃P)₂PdCl₂, Cu(I)I, Et₃N, DMF, 8 h, 25° C.; (b) NaOCH₃, CH₃OH, 4 h, 25° C.; then Amberlite IR-120(plus) ion-exchange resin (H⁺ form); (c) Boc₂O, Et₃N, DMAP, THF, 12 h, 25° C.; (d) azidoethanol, Cu(I) acetate, CH₃OH, 6 h, 25° C.; (e) Ts₂O, Et₃N, DMAP, CH₂Cl₂, 3 h, −20° C.; (f) BAST, CH₂Cl₂, 1 h, −78° C., then 4 h, 25° C.; (g) TFA, 3 h, 25° C. 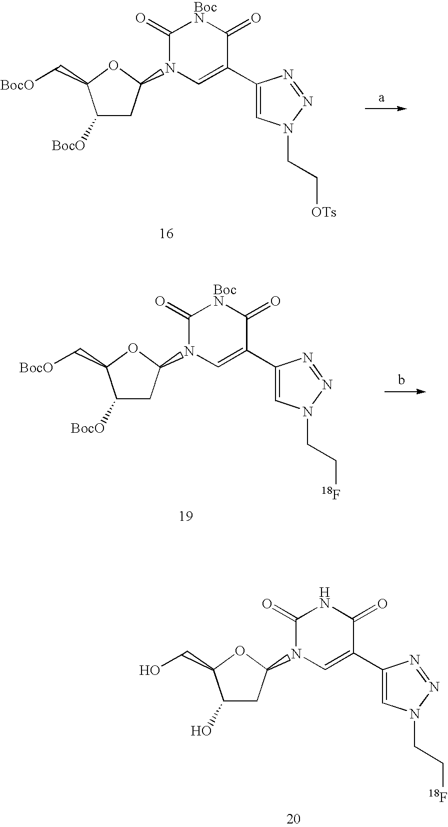
FIG. 4 | Radiosynthesis of 18F-labeled thymidine analog 20. Reagents and conditions: (a) K¹⁸F, K222/K₂CO₃, CH₃CN, 85° C., sealed vessel, 10 min; (b) 1 M HCl, 105° C., sealed vessel, 3 min.
Experiment Section:

### Synthesis of 1-((2R, 4S, 5R)-4-hydroxy-5-(hydroxymethyl)tetrahydrofuran-2-yl)-5-((trimethylsilyl)pyrimidine-2,4(1H, 3H)-dione (12)

5-Iodo-2′-deoxyuridine (5.10 g, 14.4 mmol), DMF (36 mL), Et₃N (72 mL), CuI (221 mg, 1.16 mmol), dichlorobis(triphenylphosphine)palladium(II) (233 mg, 0.33 mmol) and trimethylsilylethyne (6.93 g, 71 mmol) were added sequentially to a dried round-bottomed flask (250 mL) with stirring under Argon. The reaction was continued at room temperature for 8 h. After removing the solvent under reduced pressure, the residue was purified with column chromatography (silica gel, CH₃OH:CHCl₃ 1:9) to give the product (3.84 g, 82%). ¹H NMR (300 MHz, DMSO-d₆): δ 0.18 (s, 9H), 2.10-2.15 (m, 2H), 3.59 (ddd, 2H, J=12.0, 6.0, 3.0 Hz), 3.79 (q, 1H, J=3.0 Hz), 4.21-4.24 (m, 1H), 5.11 (t, 1H, J=6.0 Hz), 5.25 (d, 1H, J=3.0 Hz), 6.09 (t, 1H, J=6.0 Hz), 8.27 (s, 1H), 11.63 (s, 1H). ³C NMR (75 MHz, DMSO-d₆): δ 0.00, 40.39, 60.84, 69.97, 84.84, 87.62, 97.06, 98.00, 98.29, 144.74, 149.42, 161.47.

### Synthesis of 5-Ethynyl-1-((2R, 4S, 5R)-4-hydroxy-5-(hydroxymethyl)tetra-hydrofuran-2-yl)pyrimidine-2,4(1H, 3H)-dione (13)

Compound 12 (3.25 g, 10 mmol) was dissolved in MeOH (40 mL) with stirring and NaOMe (1.08 g, 20 mmol) was added. The reaction was stirred at room temperature for 4 h. The solution was then neutralized by ion exchange resin Amberlite IR-120 plus (H⁺ form), filtered, concentrated under reduced pressure and chromatographed (silica gel, MeOH/CHCl₃ 1:9) to afford the product (2.0 g, 79%). ¹H NMR (300 MHz, DMSO-d₆): δ 2.11-2.15 (m, 2H), 3.59 (ddd, 2H, J=12.0, 6.0, 3.0 Hz), 3.80 (q, 1H, J=3.0 Hz), 4.11 (s, 1H), 4.22-4.24 (m, 1H), 5.14 (t, 1H, J=6.0 Hz), 5.25 (d, 1H, J=3.0 Hz), 6.10 (t, 1H, J=6.0 Hz), 8.30 (s, 1H), 11.63 (s, 1H). ¹³C NMR (75 MHz, DMSO-d₆): δ 40.29, 60.79, 69.94, 76.38, 83.60, 84.76, 87.55, 97.53, 144.50, 149.38, 161.63. MS (m/z) (ESI): 275.2 [M+Na]⁺, 527.2 [2M+Na]⁺.

### Synthesis of tert-Butyl 3-((2R, 4S, 5R)-4-(tert-butoxycarbonyloxy)-5-((tert-butoxycarbonyloxy)methyl)tetrahydrofuran-2-yl)-5-ethynyl-2,6-dioxo-2,3-dihydro-pyrimidine-1(6H)-carboxylate (14)

To a solution of compound 13 (1.514 g, 6 mmol), DMAP (0.73 g, 6 mmol), Et₃N (5.47 g, 54 mmol), THF (75 mL) was added di-tert-butyl dicarbonate (11.79 g, 54 mmol) with venting. The reaction was stirred at room temperature for 12 h. The reaction mixture was then poured onto water and extracted into CH₂Cl₂. The combined organic phases were washed with water, dried over MgSO₄, filtered and concentrated to dryness. The crude material was purified on silica gel using CH₂Cl₂ as the eluent to provide 2.5 g (75%) of a light yellow solid. ¹H NMR (300 MHz, DMSO-d₆): δ 1.42 (d, 18H), 1.52 (s, 9H), 2.35-2.45 (m, 2H), 4.25 (m, 3H), 4.29 (s, 1H), 5.08 (d, 1H, J=6.0 Hz), 6.06 (t, 1H, J=6.0 Hz), 8.09 (s, 1H). ¹³C NMR (75 MHz, DMSO-d₆): δ 27.25, 31.25, 35.92, 65.87, 76.24, 81.12, 81.97, 82.44, 83.92, 84.88, 98.27, 144.11, 149.32, 152.02, 152.57, 161.43. MS (m/z) (ESI): 575.2 μM+Na]⁺.

### Synthesis of tert-Butyl 3-((2R, 4S, 5R)-4-(tert-butoxycarbonyloxy)-5-((tert-butoxycarbonyloxy)methyl)tetrahydrofuran-2-yl)-5-(1-(2-hydroxyethyl)-1H-1,2,3-triazol-4-yl)-2,6-dioxo-2,3-dihydropyrimidine-1(6H)-carboxylate (15)

To a round bottom flask containing compound 14 (1.5 g, 2.72 mmol), azidoethanol (40% pure, 0.89 g, 4.08 mmol), CH₃OH (45 mL) was added copper (I) acetate (0.133 g, 1.09 mmol). The reaction was stirred at room temperature for 6 h. The reaction mixture was then poured onto water and extracted into ethyl acetate. The combined organic phases were washed with water, dried over MgSO₄, filtered and concentrated to dryness. The crude material was purified on silica gel using EtOAc:Hexane (7:3) as the eluent to afford 1.08 g (62%) of a light yellow solid. ¹H NMR (300 MHz, DMSO-d₆): δ 1.42 (d, 18H), 1.52 (s, 9H), 2.31-2.39 (m, 2H), 3.77 (d, 2H), 4.12 (d, 2H), 4.23 (m, 3H), 4.44 (t, 1H), 4.65 (t, 1H), 6.16 (m, 1H), 7.94 (d, 1H), 8.33 (s, 1H). ¹³C NMR (75 MHz, DMSO-d₆): δ 27.50, 31.25, 52.10, 59.94, 66.90, 76.48, 82.44, 82.49, 86.63, 105.73, 110.85, 122.97, 138.23, 147.12, 149.53, 151.99, 161.45. MS (m/z) (ESI): 640.2 μM+H]⁺.

### Synthesis of tert-Butyl 3-((2R, 4S, 5R)-4-(tert-butoxycarbonyloxy)-5-((tert-butoxycarbonyloxy)methyl)tetrahydrofuran-2-yl)-2,6-dioxo-5-(1-(2-tosyloxy)ethyl)-1H-1,2,3-triazol-4-yl)-2,3-dihydropyrimidine-1(6H)-carboxylate (16)

To a solution of compound 15 (0.4 g, 0.626 mmol), DMAP (8 mg, 0.06 mmol), Et₃N (0.634 g, 6.26 mmol), and CH₂Cl₂ (8 mL) at −20° C. was added p-toluenesulfonic anhydride (0.817 g, 2.5 mmol). The reaction was stirred at −20° C. for 3 h. The reaction mixture was then poured onto water and extracted into CH₂Cl₂. The combined organic phases were dried over MgSO₄, filtered and concentrated to dryness. The crude material was purified on silica gel by elution with EtOAc:Hexane (3:2) to provide 0.38 g (77%) of a light yellow solid. ¹H NMR (300 MHz, DMSO-d₆): δ 1.35 (s, 9H), 1.45 (s, 9H), 1.56 (s, 9H), 2.62-2.75 (m, 2H), 2.34 (s, 3H), 4.26 (m, 3H), 4.43 (s, 2H), 4.68 (s, 2H), 5.14 (s, 1H), 6.19 (t, 1H, J=6.0 Hz), 7.35 (d, 2H, J=6.0 Hz), 7.58 (d, 2H, J=6.0 Hz), 8.28 (s, 1H), 8.40 (s, 1H). ¹³C NMR (75 MHz, DMSO-d₆): δ 20.98, 23.88, 27.16, 52.82, 60.12, 66.05, 76.48, 81.12, 81.88, 82.46, 125.46, 126.69, 127.40, 127.60, 128.04, 129.76, 130.22, 137.66, 145.53, 149.52, 152.07, 152.59. MS (m/z) (ESI): 794.2 [M+H]⁺.

### Synthesis of tert-Butyl 3-((2R, 4S, 5R)-4-(tert-butoxycarbonyloxy)-5-((tert-butoxycarbonyloxy)methyl)tetrahydrofuran-2-yl)-5-(1-(2-fluoroethyl)-1H-1,2,3-triazol-4-yl)-2,6-dioxo-2,3-dihydropyrimidine-1(6H)-carboxylate (17)

To a round bottom flask at −78° C. containing compound 15 (0.4 g, 0.626 mmol) in CH₂Cl₂ (10 mL) was added bis(2-methoxyethyl)aminosulfur trifluoride (0.277 g, 1.251 mmol). The reaction was stirred for 1 h, and then warmed up to room temperature for 4 h. The reaction mixture was then poured onto saturated NaHCO₃ solution and extracted into CH₂Cl₂. The combined organic phases were dried over MgSO₄, filtered and concentrated to dryness. The crude material was purified on silica gel using EtOAc:Hexane (1:1) as the eluent to afford 0.26 g (65%) of a white solid. ¹H NMR (300 MHz, DMSO-d₆): δ 1.35 (s, 9H), 1.45 (s, 9H), 1.54 (s, 9H), 2.55-2.70 (m, 2H), 4.20 (m, 1H), 4.31 (d, 2H), 4.77 (d, 2H), 4.81 (t, 1H), 4.91 (t, 1H), 5.12 (t, 1H), 6.16 (t, 1H), 8.41 (d, 1H), 8.47 (s, 1H). ¹³C NMR (75 MHz, DMSO-d₆): δ 27.14, 36.17, 58.13, 65.98, 76.48, 81.16, 81.85, 82.45, 86.64, 105.48, 110.85, 122.91, 135.74, 138.68, 149.52, 152.51, 152.58, 161.08. ¹⁹F NMR (282 MHz, DMSO-d₆): δ−222.22. MS (m/z) (ESI): 642.2 [M+H]⁺.

### Synthesis of 5-(1-(2-fluoroethyl)-1H-1,2,3-triazol-4-yl)-1-((2R, 4S, 5R)-4-hydroxy-5-(hydroxymethyl)tetrahydrofuran-2-yl)pyrimidine-2,4(1H, 3H)-dione (18)

To a round bottom flask containing compound 17 (0.2 g, 0.31 mmol) was added trifluoroacetic acid (3 mL). The reaction was stirred at room temperature for 3 h. The reaction was then concentrated to dryness and the residue was purified on silica gel using CH₂Cl₂:CH₃OH (4:1) as the eluent to afford 65 mg (61%) of a white solid. ¹H NMR (300 MHz, DMSO-d₆): δ 2.16-2.18 (m, 2H), 3.57 (s, 2H), 3.59 (s, 1H), 3.83 (dd, 2H, J=6.0 Hz), 4.26 (dd, 1H, J=6.0 Hz), 4.73 (m, 2H), 4.79 (dd, 1H), 4.90 (dd, 1H), 6.24 (t, 1H, J=9.0 Hz), 8.40 (s, 1H), 8.51 (s, 1H). ¹³C NMR (75 MHz, DMSO-d₆): δ 49.75, 50.01, 61.42, 70.61, 80.90, 83.13, 84.63, 87.44, 105.07, 122.69, 135.74, 139.48, 150.68. ¹⁹F NMR (282 MHz, DMSO-d₆): δ−222.06. MS (m/z) (ESI): 342.1 [M+H]⁺, 364.1 [M+Na]⁺.
Click F-18 5-Triazole Experimental

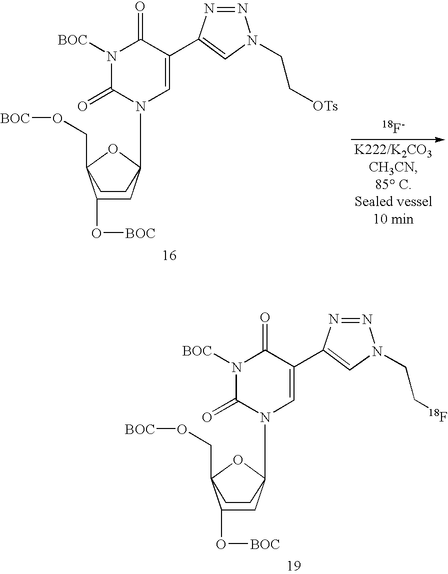

Oxygen-18 water (>97% enriched) was irradiated using 11 MeV protons (RDS-111 Eclipse, Siemens Molecular Imaging) to generate [¹⁸F]fluoride ion in the usual way. At the end of the bombardment, the [¹⁸O]water containing [¹⁸F]fluoride ion was delivered from the tantalum target to an automated nucleophilic fluorination module (explora RN, Siemens Biomarker Solutions). Under computer control, the [¹⁸O]water/[¹⁸F]fluoride ion solution was transferred by vacuum to a anion exchange resin column (Macherey-Nagel Chromafix 45-PS-HCO3⁻) which had previously been rinsed with water (5 mL), aqueous potassium bicarbonate (0.5 M, 5 mL), and water (5 mL). The [¹⁸O]water (2.0 mL) was recovered for re-use. The trapped [¹⁸F]fluoride ion was eluted into the reaction vessel with a solution of potassium carbonate (3.0 mg) in water (0.4 mL). A solution of Kryptofix® 222 (K222, 20 mg) in acetonitrile (1.0 mL) was added, and the mixture was heated (70 to 95° C.) under vacuum and a stream of argon to evaporate the acetonitrile and water. After cooling, to the residue of “dry” reactive [¹⁸F]fluoride ion, K222, and potassium carbonate, was added a solution of 5-[1-(2′-p-toluenesulfonyloxy)ethyl)-1H-[1,2,3]triazol-4-yl]-3-N-Boc-3′-O-Boc-5′-O-Boc-thymidine (“5-triazole-thymidine-tosylate”) (16, 20.9 mg, 26.3 μmol) in acetonitrile (0.9 mL). The reaction mixture was heated to 85° C. in a sealed vessel (Pₘₐₓ=2.1 bar) for 10 minutes with stirring (magnetic). The mixture was cooled to 55° C. and most of the acetonitrile was evaporated under vacuum and a stream of argon as before.

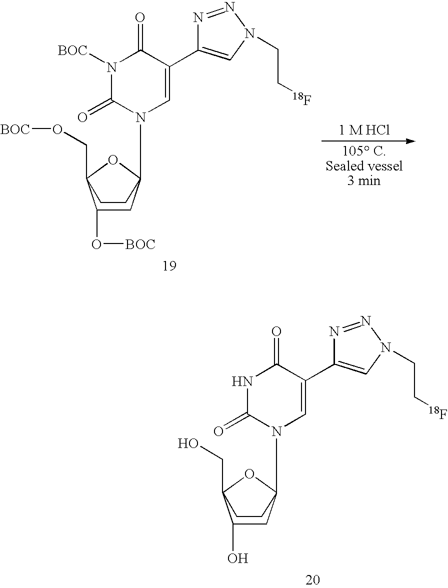

To the crude protected [¹⁸F]fluorinated intermediate (19) was added aqueous hydrochloric acid (1.0 M, 0.8 mL), and the mixture was heated to 105° C. for 3 minutes. After cooling to 35° C., aqueous sodium acetate (2.0 M, 0.4 mL) was added with stirring. The reaction mixture was transferred to a sample loop (1.5 mL), and injected onto a semi-prep HPLC column (Phenomenex Gemini 5μ C6-Phenyl, 250×10 mm, 8% ethanol, 92% 21 mM phosphate buffer pH 8.0 mobile phase, 6.0 mL/min). The product 5-[1-(2′-[¹⁸F[fluoroethyl)-1H-[1,2,3]triazol-4-yl]-thymidine (20, [¹⁸F]FETT) eluted at 14.5-15.5 minutes as monitored by UV (254 nm) and flow-through radiation detection. The HPLC eluate containing the product 20 (6-7 mL) was passed through a 0.22 μm sterile filter into a sterile vial.

A typical production run starting with about 1,001 mCi of [¹⁸F]fluoride ion gave 22.3 mCi (31.4 mCi at EOB, 3.1% yield) of isolated product after 54 minutes of synthesis and HPLC purification.

The collected product was analyzed by HPLC (Phenomenex Gemini 5μ C18, 150×4.6 mm, 10% ethanol, 90% water mobile phase, 1.0 mL/min). As monitored by radioactivity and UV (267 nm) detection, this product had a retention time of 7.95 minutes and a radiochemical purity of >99.0%.

We claim:

1. A method for preparing a radioactive ligand or radioactive substrate having affinity for a target biomacromolecule, the method comprising:
(a) reacting a first compound comprising i) a first molecular structure; ii) a leaving group; iii) a first functional group capable of participating in a click chemistry reaction; and optionally, iv) a linker between the first functional group and the molecular structure, with a radioactive reagent under conditions sufficient to displace the leaving group with a radioactive component of the radioactive reagent to form a first radioactive compound;
(b) providing a second compound comprising i) a second molecular structure; ii) a second complementary functional group capable of participating in a click chemistry reaction with the first functional group, wherein the second compound optionally comprises a linker between the second compound and the second functional group;
(c) reacting the first functional group of the first radioactive compound with the complementary functional group of the second compound via a click chemistry reaction to form the radioactive ligand or substrate; and
(d) isolating the radioactive ligand or substrate.

2. The method of claim 1, wherein the biomacromelecule is selected from the group consisting of enzymes, receptors, DNA, RNA, ion channels and antibodies.

3. The method of claim 1, wherein the biomacromolecule is a protein.

4. The method of claim 1, wherein the click chemistry reaction is a pericyclic reaction.

5. The method of claim 4, wherein the pericyclic reaction is a cycloaddition reaction.

6. The method of claim 4, wherein the pericyclic reaction is selected from the group consisting of a 1,3-dipolar cycloaddition reaction and a Diels-Alder reaction.

7. The method of claim 5, wherein the pericyclic reaction is a 1,3-dipolar cycloaddition reaction.

7. The method of claim 4, wherein the click chemistry reaction is a 1,3-dipolar cycloaddition reaction.

8. The method of claim 1, wherein the first functional group is an azide and the second functional group is a terminal alkyne, or wherein the first functional group is a terminal alkyne and the second functional group is an azide.

9. The method of claim 1, wherein the complementary click functional groups comprises an azide and an alkyne and the click reaction forms the radioactive ligand or substrate comprising a 1,4- or 1,5-disubstituted 1,2,3 triazole.

10. The method of claim 9, wherein the click reaction is performed in the presence of a catalyst.

11. The method of claim 10, wherein the catalyst is a Cu(I) salt or a ruthenium (II) salt.

12. The method of claim 9, wherein the click reaction is performed at slightly elevated temperatures between 25° C. and 200° C.

13. The method of claim 1, wherein the radioactive agent is a coordinating compound comprising a phase transfer catalyst and a salt complex.

14. The method of claim 1, wherein the radioactive agent is selected from the group consisting of n-Bu₄NF-F 18, Kryptofix [2,2,2] or potassium carbonate, or potassium bicarbonate, or cesium carbonate, or cesium bicarbonate and/or potassium 18F-fluoride and/or cesium 18F-fluoride.

15. The method of claim 1, wherein the displacement reaction is performed in a polar aprotic solvent selected from the group consisting of acetonitrile, acetone, 1,4-dioxane, tetrahydrofuran (THF), tetramethylenesulfone (sulfolane), N-methylpyrrolidinone (NMP), dimethoxyethane (DME), dimethylacetamide (DMA), N,N-dimethylformamide (DMF), dimethylsulfoxide (DMSO) and hexamethylphosphoramide (HMPA) and mixtures thereof, and the click reaction is performed in either polar aprotic solvents or in polar protic solvents selected from the group consisting of methanol, ethanol, 2-propanol, tertiary-butanol, n-butanol and/or water or buffered solutions thereof.

16. The method of claim 1, wherein the leaving group is selected from the group consisting of halogens, the nitro moiety, diazonium salts and sulfonate esters.

17. The method of claim 1, wherein the linker between the first functional group and the first molecular structure or the linker between the second functional group and the second molecular structure, comprises between 1 to 10 atoms in the linker chain.

18. The method of claim 1, wherein the first molecular structure or the second molecular structure is a nucleic acid derivative.

19. The method of claim 16, wherein the nucleic acid derivative is a thymidine derivative.

20. The method of claim 1, wherein the radioactive substrate is prepared according to the process scheme below: 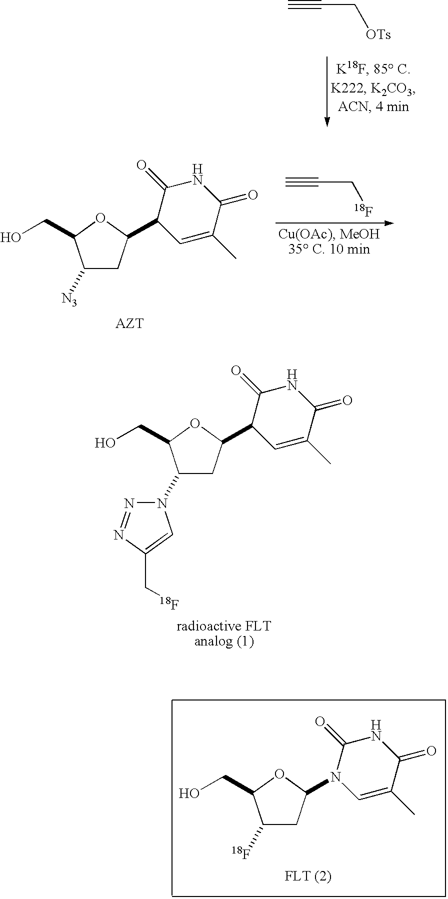
wherein the first molecular structure is des-azido AZT, the first functional group is an azide, the second molecular structure is a —CH₂— group, the leaving group attached to the second molecular structure is —OTs, and the radioactive substrate is the radioactive FLT analog.

21. The process of claim 1, wherein the substrate or ligand is prepared according to the process scheme below: 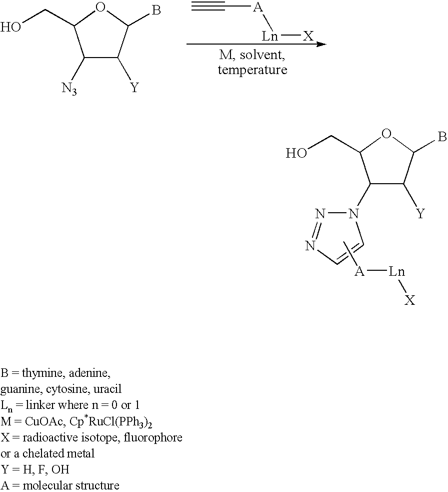
wherein:
the base (B) on the ribose ring is selected from the group consisting of adenine, guanine, cytosine, thymine and uracil;
when the catalyst is CuOAc, the reaction forms a 1,4 triazole product or when the catalyst is Cp*RuCl(PPh₃)₂, the reaction forms a 1,5-triazole product;
X is selected from the group consisting of a radioactive isotope, a fluorophore and a chelated metal; and
optionally, wherein X is attached to the alkyne via a linker.

22. A process for preparing a substrate or ligand according to the process scheme below: 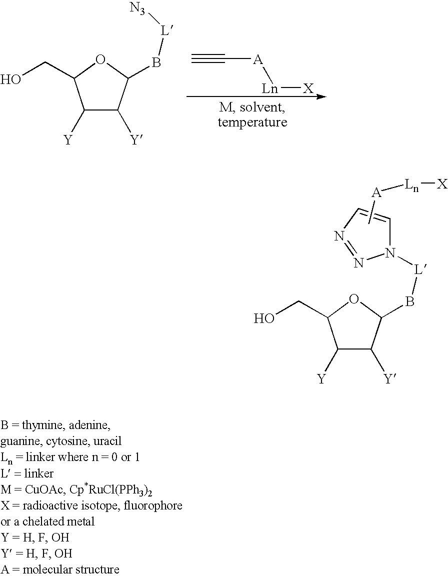
wherein:
the base (B) on the ribose ring is selected from the group consisting of adenine, guanine, cytosine, thymine and uracil, and where the base comprises an azide optionally attached to a linker L′, wherein the base are substituted and functionalized as selected from the group consisting of:
1) B=thymine, where the azide is optionally attached via a linker to the 3-position, the 5-methyl or the 6-position;
2) B=cytosine, where the azide is optionally attached via a linker to the 4-N nitrogen, the 5-position or the 6-position;
3) B=uracil, where the azide is optionally attached via a linker to the 3-N nitrogen, the 5-position or the 6-position;
4) B=adenine, where the azide is optionally attached via a linker to the 6-N nitrogen, the 2-position or the 8-position; and
5) B=guanine, where the azide is optionally attached via a linker to the 2-N nitrogen, the 1-N nitrogen or the 8-position;
wherein the catalyst is CuOAc, then the reaction forms a 1,4 triazole or where the catalyst is Cp*RuCl(PPh₃)₂, then the reaction forms a 1,5-triazole; wherein
X is the radioactive element attached to the alkyne via a linker; or
wherein X is a radioactive isotope, fluorophore or chelated metal; and
wherein Y is hydrogen, fluorine or hydroxyl.

23. A process for preparing a substrate or ligand according to the process below: 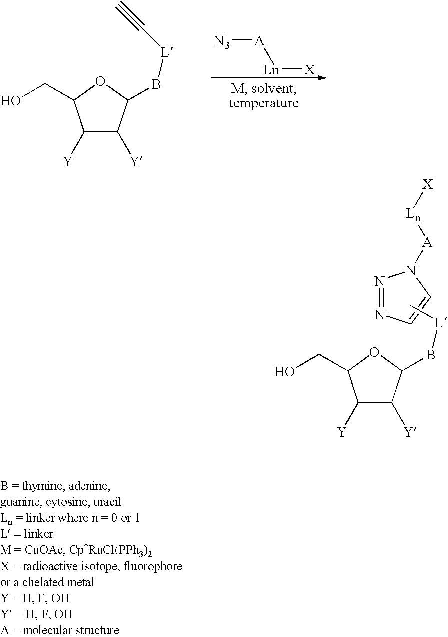
wherein:
B is a base attached to the ribose ring and is selected from the group consisting of adenine, guanine, cytosine, thymine and uracil; or
wherein B=thymine and the alkyne is attached optionally via a linker to the 3-position, the 5-methyl, or the 6-position of the ribose; or
wherein B=cytosine and the alkyne is attached optionally via a linker to the 4-N nitrogen, the 5-position or the 6-position; or
wherein B=uracil and the alkyne is attached optionally via a linker to the 3-N nitrogen, the 5-position or the 6-position; or
wherein B=adenine and the alkyne is attached optionally via a linker to the 6-N nitrogen, the 2-position or the 8-position; or
wherein B=guanine and the alkyne is attached optionally via a linker to the 2-N nitrogen, the 1-N nitrogen or the 8-position; and
where the catalyst is CuOAc, the reaction forms a 1,4 triazole, or when the catalyst is Cp*RuCl(PPh₃)₂ the reaction forms a 1,5-triazole; or
wherein X is a radioactive isotope, fluorophore or chelated metal; and
Y is hydrogen, fluorine or hydroxyl.

24. A method for preparing a radioactive ligand or substrate having affinity for a target biomacromolecule, the method comprising:
(a) providing a first compound comprising i) a first molecular structure; ii) a leaving group; iii) a first functional group capable of participating in a click chemistry reaction; and optionally, iv) a linker between the first functional group and the molecular structure;
(b) providing a second compound comprising i) a second molecular structure; ii) a second complementary functional group capable of participating in a click chemistry reaction with the first functional group, wherein the second compound optionally comprises a linker between the second compound and the second functional group;
(c) reacting the first functional group with the complementary functional group of the second compound via a click chemistry reaction to form the ligand or substrate; and
(d) reacting the ligand or substrate with a radioactive reagent under conditions sufficient to displace the leaving group with a radioactive component of the radioactive reagent to form the radioactive ligand or substrate; and
(e) isolating the radioactive ligand or substrate.

25. The method of claim 24, wherein the biomacromelecule is selected from the group consisting of enzymes, receptors, DNA, RNA, ion channels and antibodies.

26. The method of claim 24, wherein the biomacromolecule is a protein.

27. The method of claim 24, wherein the click chemistry reaction is a pericyclic reaction.

28. The method of claim 27, wherein the pericyclic reaction is a cycloaddition reaction.

29. The method of claim 27, wherein the pericyclic reaction is selected from the group consisting of a 1,3-dipolar cycloaddition reaction and a Diels-Alder reaction.

30. The method of claim 29, wherein the pericyclic reaction is a Diels-Alder reaction.

31. The method of claim 29, wherein the pericyclic reaction is a 1,3-dipolar cycloaddition reaction.

32. The method of claim 24, wherein the first functional group is an azide and the second functional group is an alkyne, or wherein the first functional group is an alkyne and the second functional group is an azide.

33. The method of claim 24, wherein the complementary click functional groups comprises an azide and an alkyne and the click reaction forms the radioactive ligand or substrate comprising a 1,4- or 1,5-disubstituted 1,2,3 triazole.

34. The method of claim 32, wherein the click reaction is performed in the presence of a catalyst.

35. The method of claim 34, wherein the catalyst is a Cu(I) salt or a ruthenium (II) salt.

36. The method of claim 33, wherein the click reaction is performed at slightly elevated temperatures between 25° C. and 200° C.

37. The method of claim 24, wherein the radioactive agent is a coordinating compound comprising a phase transfer catalyst and a salt complex.

38. The method of claim 24, wherein the radioactive agent is selected from the group consisting of n-Bu₄NF-F 18, Kryptofix [2,2,2] and potassium carbonate, potassium bicarbonate, cesium carbonate, cesium bicarbonate and/or potassium 18F-fluoride and/or cesium 18-F-fluoride.

39. A method for preparing a labeled biomacromolecule, the method comprising:
(a) reacting a first compound comprising i) a first molecular structure; ii) a leaving group; iii) a first functional group capable of participating in a click chemistry reaction; and optionally, iv) a linker between the first functional group and the molecular structure, with a radioactive reagent under conditions sufficient to displace the leaving group with a radioactive component of the radioactive reagent to form a first radioactive compound;
(b) providing a second compound comprising i) a macromolecule; ii) a second complementary functional group capable of participating in a click chemistry reaction with the first functional group, wherein the biomacromolecule optionally comprises a linker between the biomacromolecule and the second functional group;
(c) reacting the first functional group of the first radioactive compound with the complementary functional group of the biomacromolecule via a click chemistry reaction to form the radioactive biomacromolecule; and
(d) isolating the radioactive biomacromolecule.

40. The method of claim 39, wherein the biomacromelecule is selected from the group consisting of enzymes, receptors, DNA, RNA, ion channels and antibodies.

41. The method of claim 39, wherein the biomacromolecule is a protein.

42. The method of claim 41, wherein the protein is epidermal growth factor (EGF).

43. A method for preparing a radioactive ligand or substrate, the method comprising:
(a) providing a first compound comprising i) a first molecular structure; ii) a leaving group; iii) a first functional group capable of participating in a click chemistry reaction; and optionally, iv) a linker between the first functional group and the molecular structure;
(b) providing a second compound comprising i) a biomacromolecule; ii) a second complementary functional group capable of participating in a click chemistry reaction with the first functional group, wherein the second compound optionally comprises a linker between the biomacromolecule and the second functional group;
(c) reacting the first functional group with the complementary functional group of the second compound via a click chemistry reaction to form the ligand or substrate; and
(d) reacting the ligand or substrate with a radioactive reagent under conditions sufficient to displace the leaving group with a radioactive component of the radioactive reagent to form the radioactive ligand or substrate; and
(e) isolating the radioactive ligand or substrate.

44. The method of claim 43, wherein the biomacromelecule is selected from the group consisting of enzymes, receptors, DNA, RNA, ion channels and antibodies.

45. The method of claim 43, wherein the biomacromolecule is a protein.

46. The method of claim 43, wherein the leaving group is selected from the group consisting of halogens, the nitro moiety, diazonium salts and sulfonate esters.
